(19) United States
(12) Patent Application Publication
(10) Pub. No.: US 2023/0177679 A1
(43) Pub. Date: Jun. 8, 2023

(54) IMAGE PROCESSING APPARATUS, IMAGE PROCESSING METHOD, AND IMAGE PROCESSING SYSTEM

(71) Applicant: Sony Group Corporation, Tokyo (JP)

(72) Inventor: Hisakazu SHIRAKI, Tokyo (JP)

(73) Assignee: Sony Group Corporation, Tokyo (JP)

(21) Appl. No.: 17/917,274

(22) PCT Filed: Apr. 19, 2021
(86) PCT No.: PCT/JP2021/015806

§ 371 Date: Oct. 6, 2022

(30) Foreign Application Priority Data
Apr. 27, 2020 (JP) 2020-078332

(51) Int. Cl. G06T 7/00; G06T 3/40; G06T 5/40; G06V 10/143; G06V 10/56; G06V 10/70

(52) U.S. Cl. CPC G06T 7/0012; G06T 3/40; G06T 5/40; G06V 10/56; G06V 10/70; G06V 10/143; G06T 2207/10024; G06T 2207/10056; G06T 2207/30024

(57) ABSTRACT

An image processing apparatus includes a first pixel signal acquisition unit that acquires a first pixel signal by photographing of a region of a biological tissue specimen, a range acquisition unit that acquires a pixel value range in the first pixel signal, a number-of-times determination unit that determines the number of times of photographing for at least a part of the region to be photographed of the biological tissue specimen based on the pixel value range, a second pixel signal acquisition unit that acquires a second pixel signal, which is a pixel signal of at least a part of the region to be photographed of the biological tissue specimen, the second pixel signal being obtained by being photographed according to the number of times of photographing, and an image generation unit that generates an output image based on at least a part of a plurality of the second pixel signals.

### FIELD

The present disclosure relates to an image processing apparatus, an image processing method, and an image processing system.

### BACKGROUND

In recent years, a technology for utilizing, for pathological diagnosis or the like, a digital image of a biological tissue specimen stained with a staining reagent and acquired by a digital imaging technology has been developed. In order to appropriately perform pathological diagnosis, the image is required to be a clear image in which details of the biological tissue specimen are held as they are even if a stained state varies so that information necessary for diagnosis is not overlooked. Examples of such a technique include a technique disclosed in Patent Document 1 below. Specifically, in the technique disclosed in Patent Literature 1 below, a clear image of a biological tissue specimen is acquired by correcting, based on spectral characteristic information of the biological tissue specimen, an image of the biological tissue specimen having variations in a stained state according to the stained state.

### CITATION LIST

### Patent Literature

Patent Literature 1: JP 2012 -78156 A

### SUMMARY

### Technical Problem

However, in the technique disclosed in Patent Literature 1, the spectral characteristic information is acquired using a multi-spectral sensor and the correction is performed based on the spectral characteristic information. Therefore, it is requested to provide the multi-spectral sensor in a photographing device. Therefore, in the technique of Patent Literature 1 described above, it is difficult to suppress an increase in manufacturing cost of the photographing device, and there is a possibility that an increase in the size of the photographing device is caused.

Therefore, the present disclosure proposes an image processing apparatus, an image processing method, and an image processing system that can acquire a clear digital image of a biological tissue specimen while avoiding an increase in manufacturing cost and an increase in size.

### Solution to Problem

According to the present disclosure, an image processing apparatus is provided. The image processing apparatus includes: a first pixel signal acquisition unit that acquires a first pixel signal by photographing of a region to be photographed of a biological tissue specimen; a range acquisition unit that acquires a pixel value range in the first pixel signal; a number-of-times determination unit that determines a number of times of photographing for at least a part of the region to be photographed of the biological tissue specimen based on the pixel value range; a second pixel signal acquisition unit that acquires a second pixel signal, which is a pixel signal of at least a part of the region to be photographed of the biological tissue specimen, the second pixel signal being obtained by being photographed according to the number of times of photographing; and an image generation unit that generates an output image based on at least a part of a plurality of the second pixel signals.

Also, according to the present disclosure, an image processing method is provided. The image processing method includes: acquiring a first pixel signal by photographing of a region to be photographed of a biological tissue specimen; acquiring a pixel value range in the first pixel signal; determining a number of times of photographing for at least a part of the region to be photographed of the biological tissue specimen based on the pixel value range; acquiring a second pixel signal, which is a pixel signal of at least a part of the region to be photographed of the biological tissue specimen, the second pixel signal being obtained by performing photographing according to the number of times of imaging; and generating an output image based on at least part of a plurality of the second pixel signals, by an image processing apparatus.

Moreover, according to the present disclosure, an image processing system is provided. The image processing system includes: an image processing apparatus that executes image processing; and a program for causing the image processing apparatus to execute the image processing. In the image processing system, the image processing apparatus includes: a first pixel signal acquisition unit that acquires a first pixel signal by photographing of a region to be photographed of a biological tissue specimen; a range acquisition unit that acquires a pixel value range in the first pixel signal; a number-of-times determination unit that determines a number of times of photographing for at least a part of the region to be photographed of the biological tissue specimen based on the pixel value range; a second pixel signal acquisition unit that acquires a second pixel signal, which is a pixel signal of at least a part of the region to be photographed of the biological tissue specimen, the second pixel signal being obtained by being photographed according to the number of times of photographing; and an image generation unit that generates an output image based on at least a part of a plurality of the second pixel signals.

### BRIEF DESCRIPTION OF THE DRAWINGS

FIG. 1 is an explanatory diagram for explaining an overview of an embodiment of the present disclosure.

FIG. 2 is an explanatory diagram for explaining a comparison between the embodiment of the present disclosure and a comparative example.

FIG. 3 is a block diagram illustrating a configuration example of an image processing system 10 according to a first embodiment of the present disclosure.

FIG. 4 is a block diagram illustrating a configuration example of an image processing apparatus 200 according to the embodiment.

FIG. 5 is an explanatory diagram for explaining divided regions 500 according to the embodiment.

FIG. 6 is a flowchart illustrating an example of image processing according to the embodiment.

FIG. 7 is an explanatory diagram for explaining image processing according to the embodiment.

FIG. 8 is a block diagram illustrating a configuration example of an image processing apparatus 200a according to a second embodiment of the present disclosure.

FIG. 9 is a flowchart illustrating an example of image processing according to the embodiment.

FIG. 10 is an explanatory diagram for explaining the image processing according to the embodiment.

FIG. 11 is a block diagram illustrating a configuration example of an image processing system 10b according to a third embodiment of the present disclosure.

FIG. 12 is a block diagram illustrating a configuration example of an image processing apparatus 200b according to the embodiment.

FIG. 13 is a flowchart illustrating an example of image processing according to the embodiment.

FIG. 14 is an explanatory diagram for explaining an example of a table 252 according to the embodiment.

FIG. 15 is an explanatory diagram (part 1) for explaining the image processing according to the embodiment.

FIG. 16 is an explanatory diagram (part 2) for explaining the image processing according to the embodiment.

FIG. 17 is a block diagram illustrating a configuration example of an image processing apparatus 200c according to a fourth embodiment of the present disclosure.

FIG. 18 is a flowchart illustrating an example of image processing according to the embodiment.

FIG. 19 is an explanatory diagram for explaining an example of a table 254 according to the embodiment.

FIG. 20 is an explanatory diagram for explaining a fifth embodiment of the present disclosure.

FIG. 21 is a block diagram illustrating a configuration example of an image processing apparatus 200d according to the embodiment.

FIG. 22 is a flowchart illustrating an example of image processing according to the embodiment.

FIG. 23 is an explanatory diagram for explaining an example of a table 256 according to the embodiment.

FIG. 24 is a block diagram illustrating an example of a schematic configuration of a diagnosis support system.

FIG. 25 is a hardware configuration diagram illustrating an example of a computer 1000 that realizes functions of the image processing apparatus 200.

### DESCRIPTION OF EMBODIMENTS

Preferred embodiments of the present disclosure are explained in detail below with reference to the accompanying drawings. Note that, in the present specification and the drawings, components having substantially the same functional configurations are denoted by the same reference numerals and signs, whereby redundant explanation of the components is omitted. In addition, in the present specification and the drawings, a plurality of components having substantially the same or similar functional configurations are sometimes distinguished by attaching different alphabets after the same reference numerals. However, when it is not particularly necessary to distinguish each of the plurality of components having substantially the same or similar functional configurations, only the same reference numerals and signs are attached.

Note that the explanation is made in the following order.
1. Overview of embodiments of the present disclosure
2. First Embodiment
   2.1 Image processing system
   2.2 DPI scanner
   2.3 Image processing apparatus
   2.4 Image processing method
   2.5 Modification
3. Second Embodiment
   3.1 Image processing apparatus
   3.2 Image processing method
   3.3 Modification
4. Third Embodiment
   4.1 Image processing system
   4.2 Image processing apparatus
   4.3 Image processing method
5. Fourth Embodiment
   5.1 Image processing apparatus
   5.2 Image processing method
6. Fifth Embodiment
   6.1 Image processing apparatus
   6.2 Image processing method
   6.3 Modification
7. Summary
8. Application Examples
9. Hardware configuration
10. Supplement

### 1. Overview of Embodiments of the Present Disclosure

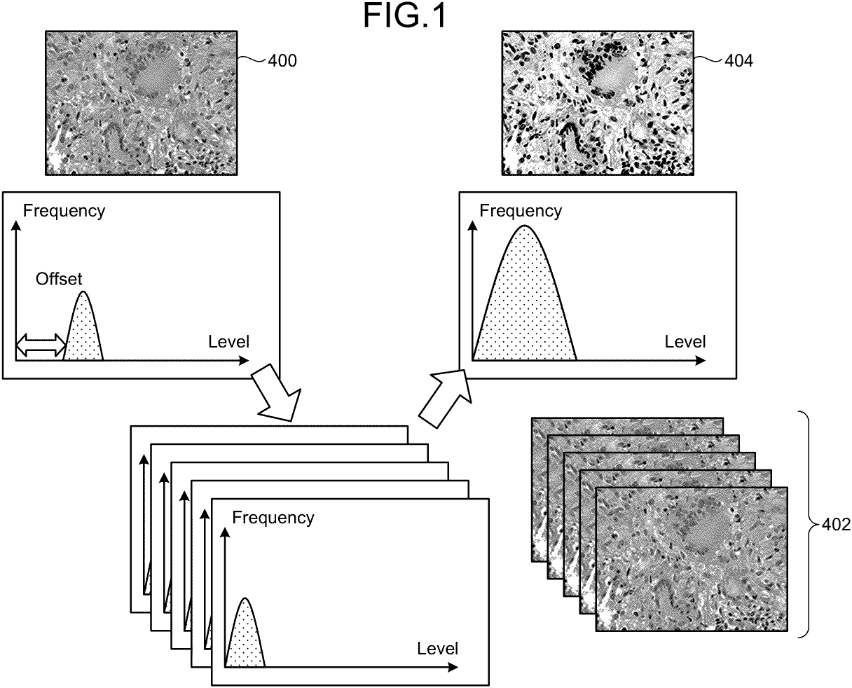

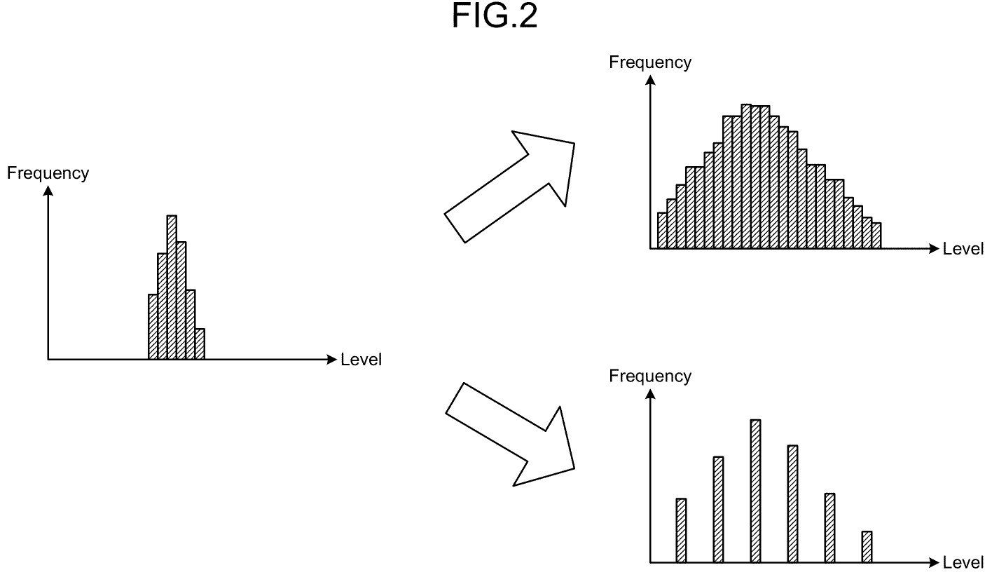

First, before explaining details of embodiments of the present disclosure, a background leading to creation of the embodiments of the present disclosure by the present inventor and an overview of the embodiments of the present disclosure are explained with reference to FIG. 1 and FIG. 2. FIG. 1 is an explanatory diagram for explaining an overview of an embodiment of the present disclosure and FIG. 2 is an explanatory diagram for explaining a comparison between the embodiment of the present disclosure and a comparative example. Note that, here, the comparative example means image processing that the inventor had repeatedly studied before the inventor created the embodiment of the present disclosure.

In pathological diagnosis and the like, analysis is sometimes performed on an image obtained by observing, with a microscope or a digital imaging system, a biological tissue specimen taken out from an organism. In order to appropriately perform pathological diagnosis, such an image is requested to be an image in which details of the biological tissue specimen are held as they are so that information necessary for diagnosis is not overlooked. However, since the details of the biological tissue specimen are easily deteriorated in a digital image compared with an optical image conventionally used for pathological diagnosis, the digital image is rarely used in the pathological diagnosis, although image management and the like are easy.

In observation of the biological tissue specimen, staining of the biological tissue specimen by a staining reagent is often performed beforehand in order to facilitate the observation of the biological tissue specimen. The staining means fixing a dye to the biological tissue specimen with a chemical reaction. It is difficult to uniformly perform the staining in one biological tissue specimen or in a plurality of biological tissue specimens. However, if there is a variation in a stained state of the biological tissue specimen, it is difficult to capture details of the biological tissue specimen with the digital image. Therefore, when the pathological diagnosis is performed using the digital image of the biological tissue specimen, information necessary for the diagnosis in order to appropriately perform the pathological diagnosis is likely to be overlooked.

Therefore, in order to avoid deterioration of the details even in a stained biological tissue specimen, it has been studied to perform image processing on the digital image used for the pathological diagnosis as explained above to obtain a clear image having high contrast. A case in which such image processing is performed is referred to as a comparative example and details of the comparative example are explained below.

Specifically, in the comparative example, an image having a distribution of pixel values (pixel signals) (for example, luminance values) illustrated on the left side of FIG. 2 is acquired and general image processing (offset correction, gain correction, and the like) is performed on the acquired image. Then, in the comparative example, an image having a distribution of pixel values illustrated in the lower right of FIG. 2 can be obtained by the image processing. In the image obtained in the comparative example, as illustrated in the lower right of FIG. 2, contrast is increased by expanding a gradation width (In the following explanation, referred to as dynamic range), which is width in which luminance values are distributed. However, in the comparative example, as it is seen from the lower right figure of FIG. 2, there is a high possibility that information is missing because of insufficient gradation (discontinuity) involved in the expansion of the dynamic range and deterioration of details occurs because of appearance of a false contour or the like. In the comparative example, since noise of an image acquired first is maintained as it is, the noise is sometimes more emphasized by expanding the dynamic range. That is, in the comparative example, since the deterioration of the details and the emphasis of the noise can occur, it is difficult to say that a digital image suitable as an image used for pathological diagnosis can be obtained.

Therefore, in view of such a situation, the present inventor has created an embodiment of the present disclosure. Specifically, in the embodiment of the present disclosure, first, a low-contrast image illustrated in the upper left of FIG. 1 is acquired as a primary image 400. Then, in the present embodiment, a distribution (a dynamic range) of pixel values of the primary image 400, which can be illustrated as a graph in the upper left of FIG. 1, is analyzed. The number of times of photographing N for acquiring secondary images 402 and a correction value for correcting the secondary images 402 are acquired based on an analysis result.

Subsequently, in the present embodiment, N low-contrast images illustrated in the lower part of FIG. 1 are acquired as secondary images 402 according to the number of times of photographing N. Then, by correcting pixel values of the acquired secondary images 402 with the correction value (specifically, performing correction for subtracting an offset value), pixel values of the secondary images 402 have a distribution illustrated as a graph in the lower part of FIG. 1. Further, in the present embodiment, a high-contrast composite image 404 can be obtained by adding up the corrected N secondary images 402 (specifically, the pixel values of the secondary images 402). The pixel values of the composite image 404 indicate a wide distribution as illustrated in as a graph in the upper right of FIG. 1 through the correction or the addition.

That is, in the present embodiment, as it is seen from the upper right figure of FIG. 2, even when the dynamic range is expanded, since the plurality of secondary images 402 are added up, it is possible to avoid missing of information due to insufficient gradation (discontinuity). As a result, according to the present embodiment, it is possible to suppress deterioration of the details due to lack of information, it is possible to capture details inherent in the biological tissue specimen and obtain a natural image. Further, according to the present embodiment, since the plurality of secondary images 402 are added up, noise (specifically, a ratio of noise) included in the composite image 404 can be reduced. Therefore, according to the present embodiment, a clear digital image of the biological tissue specimen can be acquired. Details of such an embodiment according to the present disclosure are sequentially explained below.

In the following explanation, a tissue slice or a cell, which is a part of a tissue (for example, an organ or an epithelial tissue) acquired from an organism (for example, a human body, a plant, or the like) is referred to as a biological tissue specimen. Note that the biological tissue specimen explained below may be applied with various types of staining according to necessity. In other words, in embodiments explained below, various types of staining may not be applied to the biological tissue specimen unless particularly noted otherwise. Further, for example, the staining includes not only general staining represented by hematoxylin-eosin (HE) staining, Giemsa staining, or Papanicolaou staining but also periodic acid-Schiff (PAS) staining or the like used when focusing on a specific tissue and fluorescent staining such as FISH (Fluorescence In-Situ Hybridization) or an enzyme antibody method.

### 2. First Embodiment

### 2.1 Information Processing System

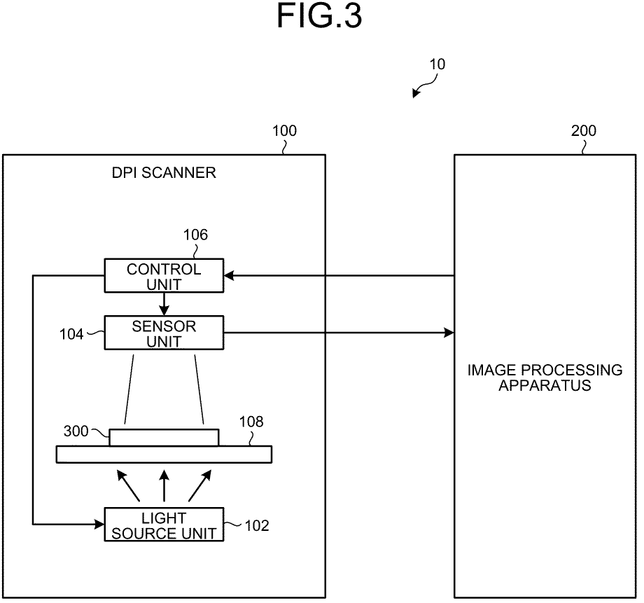

First, a configuration example of an image processing system 10 according to a first embodiment of the present disclosure is explained with reference to FIG. 3. FIG. 3 is a block diagram illustrating a configuration example of the image processing system 10 according to the first embodiment of the present disclosure. The image processing system 10 according to the present embodiment is a DPI (Digital Pathology Imaging) scanner system that performs digital photographing on a slide 300 on which a biological tissue specimen (for example, a cell tissue) is mounted.

As illustrated in FIG. 3, the image processing system 10 according to the present embodiment can include a DPI scanner (a photographing unit) 100 and an image processing apparatus 200. Note that the DPI scanner 100 and the image processing apparatus 200 may be communicably connected to each other via various wired or wireless communication networks. In addition, the DPI scanners 100 and the image processing apparatuses 200 included in the image processing system 10 according to the present embodiment are not limited to numbers illustrated in FIG. 3 and more DPI scanners 100 and image processing apparatuses 200 may be included. Further, the image processing system 10 according to the present embodiment may include not-illustrated other servers, apparatuses, and the like. An overview of the apparatuses included in the image processing system 10 according to the present embodiment is explained below.

### DPI Scanner 100

The DPI scanner 100 irradiates the slide 300 of the biological tissue specimen placed on a stage 108 of the DPI scanner 100 with predetermined illumination light and can photograph (image) light transmitted through the slide 300, light emission from the slide 300, or the like. For example, the DPI scanner 100 includes a magnifying glass (not illustrated), a digital camera (not illustrated), and the like that can enlarge and photograph a biological tissue specimen. Note that the DPI scanner 100 may be realized by any device having a photographing function, such as a smartphone, a tablet, a game machine, or a wearable device, for example. Further, the DPI scanner 100 is controlled to be driven by an image processing apparatus 200 explained below. An image photographed by the DPI scanner 100 is stored in, for example, the image processing apparatus 200. Note that a detailed configuration of the DPI scanner 100 is explained below.

### Image Processing Apparatus 200

The image processing apparatus 200 is an apparatus having a function of controlling the DPI scanner 100 and processing an image photographed by the DPI scanner 100. Specifically, the image processing apparatus 200 controls the DPI scanner 100, photographs a digital image of a biological tissue specimen, and carries out predetermined image processing on the obtained digital image. The image processing apparatus 200 is realized by any apparatus having a control function and an image processing function such as a PC (Personal Computer), a tablet, and a smartphone. Note that a detailed configuration of the image processing apparatus 200 is explained below.

Note that, in the present embodiment, the DPI scanner 100 and the image processing apparatus 200 may be an integrated apparatus, that is, may not be realized by a single apparatus. In the present embodiment, each of the DPI scanner 100 and the image processing apparatus 200 explained above may be realized by a plurality of apparatuses connected via various wired or wireless communication networks and cooperating with each other. Further, the image processing apparatus 200 explained above can be realized by, for example, a hardware configuration of a computer 1000 explained below.

### 2.2 DPI Scanner

Subsequently, a detailed configuration of the DPI scanner 100 according to the present embodiment is explained with reference to FIG. 3. As illustrated in FIG. 3, the DPI scanner 100 can mainly include a light source unit 102, a sensor unit 104, a control unit 106, and a stage 108. Functional blocks of the DPI scanner 100 are sequentially explained below.

### Light Source Unit 102

The light source unit 102 is an illumination device that is provided on a surface side of the stage 108 opposite to a slide arrangement surface, on which the slide 300 can be arranged, and can irradiate the slide 300 of the biological tissue specimen with illumination light according to control of the control unit 106 explained below. The light source unit 102 may include, for example, a condenser lens (not illustrated) that collects the illumination light emitted from the light source unit 102 and guides the illumination light to the slide 300 on the stage 138.

### Sensor Unit 104

The sensor unit 104 is a color sensor that is provided on the slide arrangement surface side of the stage 108 and detects light of red (R), green (G), and blue (B), which are the three primary colors of colors. More specifically, the sensor unit 104 can include, for example, an objective lens (not illustrated) and an imaging element (not illustrated). According to the control of the control unit 106 explained below, the sensor unit 104 can digitally photograph the biological tissue specimen and output a digital image by the photographing to the image processing apparatus 200.

Specifically, the objective lens (not illustrated) is provided on the slide arrangement surface side of the stage 108 and makes it possible to enlarge and photograph the biological tissue specimen. That is, transmitted light transmitted through the slide 300 disposed on the stage 108 is condensed by the objective lens and forms an image on the imaging element (not illustrated) provided behind the objective lens (in other words, a traveling direction of the illumination light).

An image of a photographing range having a predetermined lateral width and a predetermined longitudinal width on the slide arrangement surface of the stage 108 is formed on the imaging element (not illustrated) according to a pixel size of the imaging element and magnification of the objective lens (not illustrated). Note that, when a part of the biological tissue specimen is enlarged by the objective lens, the photographing range is a range sufficiently narrower compared with the photographing range of the imaging element. More specifically, the imaging element can be realized by an imaging element such as a CCD (Charge Coupled Device) or a CMOS (Complementary Metal Oxide Semiconductor).

Note that, in the present embodiment, the sensor unit 104 may directly photograph the biological tissue specimen not via the objective lens or the like or may photograph the biological tissue specimen via the objective lens or the like and is not particularly limited.

### Control Unit 106

The control unit 106 can integrally control the operation of the DPI scanner 100 and includes, for example, a processing circuit realized by a CPU (Central Processing Unit), a ROM (Read Only Memory), and a RAM (Random Access Memory) For example, the control unit 106 can control the light source unit 102 and the sensor unit 104 explained above. Further, the control unit 106 may control a stage driving mechanism (not illustrated) that moves the stage 108 in various directions.

For example, the control unit 106 may control the number of times of photographing N and a photographing time of the sensor unit 104 according to a command output from the image processing apparatus 200. More specifically, the control unit 106 may control the sensor unit 104 to intermittently perform photographing with the number of times of photographing N at a predetermined interval. Further, the control unit 106 may control wavelength, irradiation intensity, or an irradiation time of the illumination light emitted from the light source unit 102. Further, the control unit 106 may control a stage driving mechanism (not illustrated) that moves the stage 108 in various directions according to a region of interest so that a preset region of interest is imaged. Note that the region of interest here means a region (a target region) that a user pays attention to for analysis and the like in the biological tissue specimen.

### Stage 108

The stage 108 is a placing table on which the slide 300 is placed. Further, the stage 108 may be provided with a stage driving mechanism (not illustrated) for moving the stage 108 in various directions. For example, by controlling the stage drive mechanism, the stage 108 can be freely moved in a direction parallel to the slide arrangement surface (an X-axis-Y-axis direction) and a direction orthogonal to the slide arrangement surface (a Z-axis direction). In the present embodiment, the stage 108 may be provided with a sample conveying device (not illustrated) that conveys the slide 300 to the stage 108. By providing such a conveyance device, the slide 300 scheduled to be photographed is automatically placed on the stage 108 and replacement of the slide 300 can be automated.

As explained above, according to the present embodiment, since it is not requested to provide a multi-spectral sensor in the DPI scanner 100, an increase in manufacturing cost and an increase in size of the image processing system 10 can be avoided.

### 2.3 Image Processing Apparatus

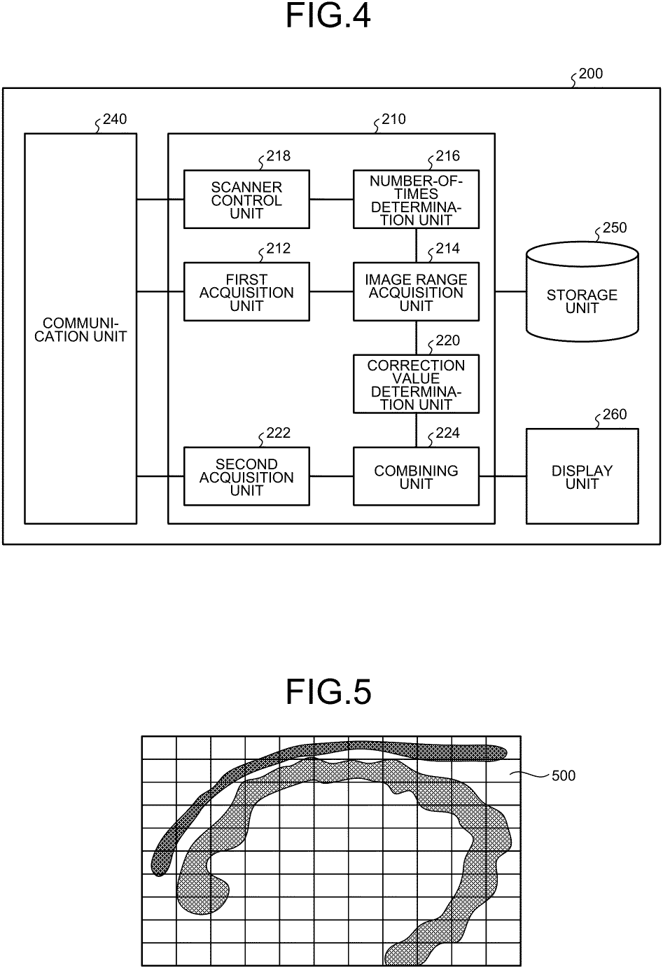

Subsequently, a detailed configuration of the image processing apparatus 200 according to the present embodiment is explained with reference to FIG. 4 and FIG. 5. FIG. 4 is a block diagram illustrating a configuration example of the image processing apparatus 200 according to the present embodiment FIG. 5 is an explanatory diagram for explaining divided regions (regions to be photographed) 500 according to the present embodiment. As explained above, the image processing apparatus 200 is the apparatus having the function of controlling the DPI scanner 100 and processing a digital image photographed by the DPI scanner 100. As illustrated in FIG. 4, the image processing apparatus 200 can mainly include a processing unit 210, a communication unit 240, a storage unit 250, and a display unit 260. Functional blocks of the image processing apparatus 200 are sequentially explained below.

### Processing Unit 210

The processing unit 210 can control the DPI scanner 100 and process a digital image received from the DPI scanner 100 and is realized by a processing circuit such as a CPU. Specifically, as illustrated in FIG. 4, the processing unit 210 mainly includes a first acquisition unit (first pixel signal acquisition unit) 212, an image range acquisition unit (a range acquisition unit) 214, a number-of-times determination unit 216, a scanner control unit (control unit) 218, a correction value determination unit 220, a second acquisition unit (a second pixel signal acquisition unit) 222, and a combining unit (an image generation unit) 224. The functional units of the processing unit 210 are sequentially explained below.

The first acquisition unit 212 acquires a pixel value (a first pixel signal) of a low-contrast primary image 400 of a region to be photographed of a biological tissue specimen from the DPI scanner 100 and outputs the pixel value to the image range acquisition unit 214 explained below. The primary image 400 acquired by the first acquisition unit 212 in this way is used when determining the number of times of photographing N for the secondary images (second photographed images) 402 acquired by the second acquisition unit 222 explained below and a correction value for correcting the secondary images 402.

The image range acquisition unit 214 acquires a dynamic range (a pixel value range), which is a distribution width of pixel values (pixel signals), in the primary image 400 received from the first acquisition unit 212 explained above. For example, the image range acquisition unit 214 acquires a level value (for example, luminance (hereinafter referred to as RGB values)) for each of colors (red, green, blue) of pixels included in the primary image 400 as a pixel value and acquires information concerning a dynamic range, which is a distribution width of RGB values (that is, an R value (a luminance value of red light), a G value (a luminance value of green light), and a B value (a luminance value of blue light)) in the primary image 400. In other words, since each of a plurality of pixels included in the primary image 400 has the R value, the G value, and the B value, the image range acquisition unit 214 acquires a minimum value to a maximum value of R values, a minimum value to a maximum value of G values, and a minimum value to a maximum value of B values in the entire pixels of the primary image 400. At this time, the image range acquisition unit 214 may convert the acquired RGB values into histograms that can indicate a frequency distribution of levels (obtained by dividing pixel value for each predetermined value range) for each of the colors and acquire maximum values and minimum values of the RGB values using the histograms. Then, the image range acquisition unit 214 outputs the acquired maximum values and minimum values of the RGB values to the number-of-times determination unit 216 and the correction value determination unit 220 explained below.

The number-of-times determination unit 216 determines the number of times of photographing N for at least a part of the divided regions (the regions to be photographed) 500 in the biological tissue specimen based on the dynamic range of the pixel values acquired by the image range acquisition unit 214 explained above. Specifically, as illustrated in FIG. 5, the number-of-times determination unit 216 virtually divides the biological tissue specimen into a plurality of divided regions 500 of a predetermined size and determines the number of times of photographing N for each of the divided regions 500. More specifically, the number-of-times determination unit 216 calculates the number of times of photographing N for the divided regions 500 based on the maximum values and the minimum values of the RGB values of the primary image 400 and outputs the calculated number of times of photographing N to the scanner control unit 218 explained below. Note that a specific method of determining the number of times of photographing N in the number-of-times determination unit 216 is explained below.

The scanner control unit 218 generates, based on the number of times of photographing N determined by the number-of-times determination unit 216 explained above, a command for controlling the DPI scanner 100 and controls the DPI scanner 100 via the communication unit 240 explained below. Specifically, the scanner control unit 218 controls the DPI scanner 100 to perform photographing with the number of times of photographing N for the divided regions 500 of the biological tissue specimen according to the generated command and acquire pixel values (second pixel signals) of the N secondary images 402 for the divided regions 500.

The correction value determination unit 220 determines, based on the dynamic range by the image range acquisition unit 214 explained above, a correction value for correction to be performed on the pixel values (second pixel signals) of the N secondary images 402 related to the divided regions 500 in the biological tissue specimen. Specifically, the correction value determination unit 220 calculates, based on the minimum values of the RGB values of the primary image 400, an offset value (details of the offset value are explained below) as a correction value and outputs the offset value to the combining unit 224 explained below. A specific method of determining the correction value in the correction value determination unit 220 is explained below.

The second acquisition unit 222 acquires pixel values (second pixel signals) of N low-contrast secondary images 402 photographed according to the number of times of photographing N and each including at least a part of the divided regions 500 in the biological tissue specimen and outputs the acquired pixel values to the combining unit 224 explained below. Specifically, in the present embodiment, the second acquisition unit 222 acquires N secondary images 402 for one divided region 500. Then, the N secondary images 402 of the divided regions 500 acquired by the second acquisition unit 222 are combined by the combining unit 224 explained below to be a composite image 404.

The combining unit 224 superimposes (adds) the N secondary images 402 of the divided regions 500 received from the second acquisition unit 222 explained above to generate a high-contrast composite image (output image) 404 of the divided regions 500. For example, the combining unit 224 can obtain the composite image 404 of the divided regions 500 by adding up pixel values of the same pixels of the N secondary images 402 of the divided regions 500. More specifically, since pixels of the secondary images 402 have an R value, a G value, and a B value (pixel values), the combining unit 224 can obtain one composite image 404 of the divided regions 500 concerning red by adding up the R values of the same pixels of the N secondary images 402, can obtain one composite image 404 of the divided regions 500 concerning green by adding up the G values of the same pixels of the N secondary images 402, and can obtain one composite image 404 of the divided regions 500 concerning blue by adding up the B values of the same pixels of the N secondary images 402.

Further, in the present embodiment, after correcting the pixel values of the pixels of the N secondary images 402 using the correction value received from the correction value determination unit 220 explained above, the combining unit 224 can add up the pixel values of the same pixels of the N secondary images 402 to obtain the composite image 404. More specifically, the combining unit 224 can perform correction by subtracting the offset value determined by the correction value determination unit 220 based on the minimum values of the RGB values from the RGB values of the pixels of the N secondary images 402 in the divided regions 500. Note that, in the present embodiment, the combining unit 224 is not limited to performing correction on each of the N secondary images 402 and may perform correction on the composite image (the output image) 404 obtained by superimposing the plurality of secondary images 402. Then, the combining unit 224 can output the composite image 404 to the storage unit 250 and the display unit 260 explained below. The combining unit 224 may generate a color image by superimposing one composite image 404 concerning red, one composite image 404 concerning green, and one composite image 404 concerning blue, which are related to the same divided regions 500, obtained as explained above.

### Communication Unit 240

The communication unit 240 can transmit and receive information to and from an external device such as the DPI scanner 100 and for example, can transmit a command for controlling the DPI scanner 100 to the DPI scanner 100. In other words, the communication unit 240 can be considered a communication interface having a function of transmitting and receiving data. In the present embodiment, the communication unit 240 is realized by, for example, a communication device (not illustrated) such as a communication antenna, a transmission/reception circuit, or a port.

### Storage Unit 250

The storage unit 250 stores programs, information, and the like for the processing unit 210 to execute various kinds of processing. Further, the storage unit 250 can function as, for example, a primary image accumulation unit (not illustrated) that stores the primary image 400 explained above, a secondary image accumulation unit (not illustrated) that stores the secondary image 402 explained above, or a composite image accumulation unit (not illustrated) that stores the composite image 404 explained above. Further, in another embodiment to be explained below, the storage unit 250 also functions as a tertiary image accumulation unit (not illustrated) and a final image accumulation unit (not illustrated) that store a tertiary image and a final image. The storage unit 250 is realized by, for example, a nonvolatile memory such as a flash memory or a storage device such as a HDD (Hard Disk Drive).

### Display Unit 260

The display unit 260 can display (output) the composite image 404. Specifically, the display unit 260 includes, for example, an LCD (Liquid Crystal Display) or an organic EL (Electro Luminescence) display and can output the composite image 404 and the like received from the combining unit 224 explained above. Note that, in the present embodiment, the display unit 260 may be provided to be fixed in the image processing apparatus 200 or may be detachably provided in the image processing apparatus 200.

Note that, in the present embodiment, the functional blocks included in the image processing apparatus 200 are not limited to the functional blocks illustrated in FIG. 4.

### 2.4 Image Processing Method

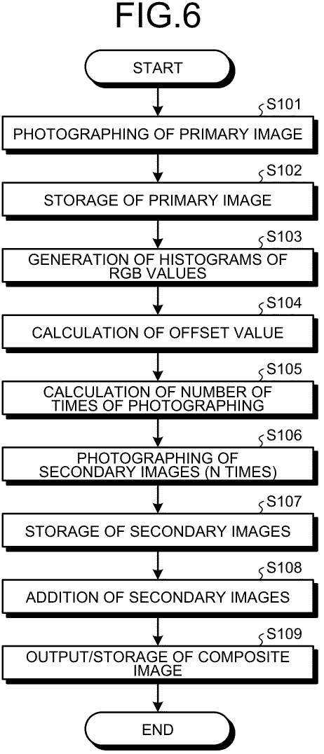

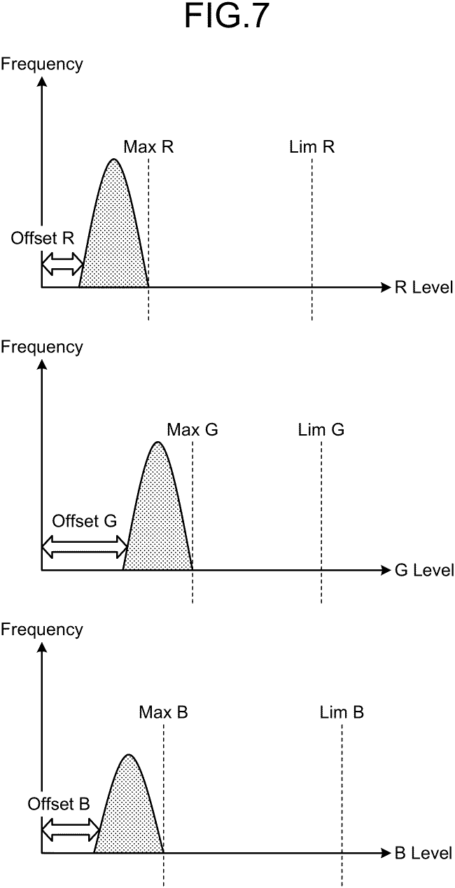

Subsequently, an image processing method according to the present embodiment is explained with reference to FIGS. 6 and 7. FIG. 6 is a flowchart illustrating an example of image processing according to the present embodiment. FIG. 7 is an explanatory diagram for explaining the image processing according to the present embodiment. Specifically, as illustrated in FIG. 6, the image processing method according to the present embodiment can include steps from step S101 to step S109. Details of these steps according to the present embodiment are explained below.

First, the image processing system 10 photographs the low-contrast primary image 400 of the entire biological tissue specimen (step S101). Subsequently, the image processing system 10 stores the primary image 400 in a primary image accumulation unit (not illustrated) of the storage unit 250 (step S102). The image processing system 10 generates histograms of RGB values from the acquired primary image 400 (step S103). The histograms generated here are, for example, histograms illustrated in FIG. 7. In FIG. 7, histograms showing distributions of luminance (RGB values) of red (R), green (G), and blue (B) are illustrated from an upper part to a lower part and, in the histograms, the luminance is divided into predetermined ranges (levels) to show frequencies of the ranges.

The image processing system 10 calculates an offset value (Offset) as a correction value (step S104). In the present embodiment, when minimum values of the luminance are represented as Offset R, Offset G, and Offset B in the histograms of the colors of the primary image 400 illustrated in FIG. 7, the offset value is a minimum value among the Offset R, Offset G, and Offset B as indicated by the following Formula (1).

$$O f f s e t = M i n i m u m \left(O f f s e t R , O f f s e t G , O f f s e t B\right)$$

Subsequently, the image processing system 10 calculates the number of times of photographing N (step S105). Specifically, in the subsequent processing, the RGB values of the pixels of the N secondary images 402 are added up. At that time, the number of times of photographing N is determined based on the following Formula (2) so that an added-up RGB value does not exceed upper limit values (Lim R, Lim G, Lim B) (see FIG. 7) of the colors set in advance according to characteristics of the sensor unit 104. Note that it is assumed that, in Formula (2), Max R, Max G, and Max B are maximum values of the RGB values in the histograms of the primary image 400 (see FIG. 7).

$$\begin{matrix}N = M i n i m u m \left(L i m R / \left(M a x R - O f f s e t\right) , L i m G / \left(M a x G - O f f s e t\right) ,\right) \\ \left(L i m B / \left(M a x B - O f f s e t\right)\right)\end{matrix}$$

The image processing system 10 photographs the low-contrast secondary image 402 of the divided regions 500 of the biological tissue specimen N times (the number of times of photographing) determined in step S105 (step S106). As explained above, in step S106, the same divided region 500 is photographed N times. Next, the image processing system 10 stores the N secondary images 402 in a secondary image accumulation unit (not illustrated) of the storage unit 250 (step S107). Note that, in the present embodiment, step S106 and step S107 may be repeatedly carried out until the N secondary images 402 can be acquired for all the divided regions 500. Alternatively, as in a modification explained below, when focusing on one or a plurality of predetermined divided regions 500, steps S106 and S107 may be performed once or repeatedly until N secondary images 402 of one or a plurality of predetermined divided regions 500 can be acquired.

According to the following Formula (3), the image processing system 10 subtracts the offset value (Offset) from the RGB values for each of the pixels of each of the N secondary images 402 in the divided regions 500 and adds up the RGB values after the subtraction for each of the same pixels to thereby acquire one composite image 404 for the colors (step S108). In Formula (3), InputImageRi (x, y), InputImageGi (x, y), and InputImageBi (x, y) are the RGB values of the pixels of the secondary images 402 for each of the colors and OutImageR (x, y), OutImageG (x, y), and OutImageB (x, y) are RGB values of each of the pixels of the composite image 404. That is, in the present embodiment, the dynamic range of the low-contrast primary image 400 is analyzed and the number of times of photographing N of the secondary image 402 and the correction value for correcting the secondary image 402 are calculated based on an analysis result, whereby suitable addition and correction can be performed. As a result, according to the present embodiment, not only the composite image 404 becomes a clear high contrast image but also black floating (saturation) in the composite image 404 can be suppressed.

$$\begin{matrix}O u t I m a g e R \left(x , y\right) = {\sum }_{i = 0}^{N} \left(I n p u t I m a g e {R}_{i} \left(x , y\right) - O f f s e t\right) \\ O u t I m a g e G \left(x , y\right) = {\sum }_{i = 0}^{N} \left(I n p u t I m a g e {G}_{i} \left(x , y\right) - O f f s e t\right) \\ O u t I m a g e B \left(x , y\right) = {\sum }_{i = 0}^{N} \left(I n p u t I m a g e {B}_{i} \left(x , y\right) - O f f s e t\right)\end{matrix}$$

In the present embodiment, by performing the addition of the N secondary images 402 together with the correction, it is possible to expand the dynamic range in the composite image 404 while reducing noise and eliminating missing of information due to insufficient gradation (discontinuity). Therefore, according to the present embodiment, a clear composite image 404 of the biological tissue specimen can be acquired.

Note that, in the present embodiment, the same number of photographing N and the same correction value (Offset value) are not limited to be applied to all the divided regions 500. An optimized number of times of photographing N and an optimized correction value may be applied to each of the divided regions 500. In such a case, in a stitching image obtained by joining the composite images 204 of the divided regions 500, in some case, a joint is discontinuous and an unnatural image is obtained. However, the contrast of images of the divided regions 500 is optimized, which is sometimes effective for analysis. In the present embodiment, the combining unit 224 may subtract noise or the like specific to the sensor unit 104 of the DPI scanner 100 at the time of the correction explained above.

Then, the image processing system 10 outputs the composite image 404 of the divided regions 500 to the display unit 260 or stores the composite image 404 in a composite image accumulation unit (not illustrated) of the storage unit 250 (step S109).

As explained above, in the present embodiment, by performing the addition of the N secondary images 402 together with the correction, it is possible to expand the dynamic range in the composite image 404 while reducing noise and eliminating missing of information due to insufficient gradation (discontinuity). Therefore, according to the present embodiment, a clear composite image 404 of the biological tissue specimen can be acquired. Furthermore, according to the present embodiment, since it is not requested to provide a multi-spectral sensor in the image processing system 10, an increase in manufacturing cost and an increase in the size of the image processing system 10 can be avoided. That is, according to the present embodiment, it is possible to acquire a clear digital image of a biological tissue specimen while avoiding an increase in manufacturing cost and an increase in size.

### 2.5 Modification

In the above explanation, it is explained that the composite image 404 of all the divided regions 500 of the biological tissue specimen is acquired. However, the present embodiment is not limited to this. For example, only the composite image 404 of a region of interest (ROI) in the biological tissue specimen may be acquired. In this way, it is possible to acquire a digital image necessary for performing analysis while reducing an image processing time. Details of such a modification are explained below.

In the present modification, after acquiring the primary image 400, the image processing system 10 outputs the acquired primary image 400 to the display unit 260 for the user. Then, the user refers to the primary image 400 (an image related to a first pixel signal) displayed on the display unit 260 and manually inputs (for example, coordinate input or surrounding by a rectangle) a range necessary for analysis in the biological tissue specimen to thereby set the region of interest. Then, in the present modification, the processing unit 210 of the image processing apparatus 200 includes a region determination unit (not illustrated) that determines one or a plurality of divided regions 500 for acquiring the secondary image 402. The region determination unit determines one or a plurality of divided regions 500 for acquiring the secondary image 402 to cover all input regions of interest. Further, the region decision unit outputs information of the determined divided regions 500 to the scanner control unit 218 and the image range acquisition unit 214. Consequently, in the present modification, only the composite image 404 of the region of interest in the biological tissue specimen can be acquired.

Note that, in the present modification, the image range acquisition unit 214 may acquire a dynamic range of pixel values for all pixels included in all the divided regions 500 of the primary image 400. Alternatively, in the present modification, the image range acquisition unit 214 may acquire a dynamic range of pixel values for pixels included in one or the plurality of divided regions 500 determined by the region determination unit in the primary image 400. Note that, in the latter case, it is possible to calculate the number of times of photographing N and a correction value suitable for the region of interest based on the dynamic range of the pixel values for the pixels included in one or a plurality of divided regions 500 determined by the region determination unit. Therefore, it is possible to acquire a clearer composite image 404 of the region of interest.

Further, the present modification is not limited to setting the region of interest by manual input of the user. The region of interest may be set based on an image recognition model obtained by machine learning. Specifically, for example, an image that can be a region of interest (for example, an image of a predetermined tissue and a predetermined cell) is subjected to machine learning in advance, feature points and feature amounts of the image that can be the region of interest are extracted, and an image recognition model is generated. Then, the region determination unit (not illustrated) can set the region of interest by extracting, from the primary image 400, an image same as or similar to the feature points or the feature amounts defined in the image recognition model. By using the machine learning in this way, since the region of interest can be automatically set, the convenience of the user can be improved and analysis can be automatically performed.

### 3. Second Embodiment

Meanwhile, in the first embodiment explained above, it is explained that RGB values are acquired as the pixel values. However, embodiments of the present disclosure are not limited to this. For example, an HLS color space for representing a color image with three values of hue (Hue), luminance (Lightness), and saturation (Saturation) may be used. That is, in a second embodiment explained below, an image is converted into an HLS signal rather than an RGB signal and hue (hereinafter referred to as H values), saturation (hereinafter referred to as S values), and luminance (hereinafter referred to as L values) of pixels are acquired as pixel values. According to the present embodiment, by using the HLS signal, the contrast in the composite image 404 can be further increased and noise can be further reduced. Details of such a second embodiment are explained below.

Note that configuration examples of the image processing system 10 and the DPI scanner 100 according to the second embodiment are common to the image processing system 10 and the DPI scanner 100 in the first embodiment. Therefore, the explanation of the configurations of the image processing system 10 and the DPI scanner 100 according to the first embodiment and FIG. 3 referred to in the explanation can be referred to. Therefore, here, explanation of the image processing system 10 and the DPI scanner 100 according to the present embodiment is omitted.

### 3.1 Image Processing Apparatus

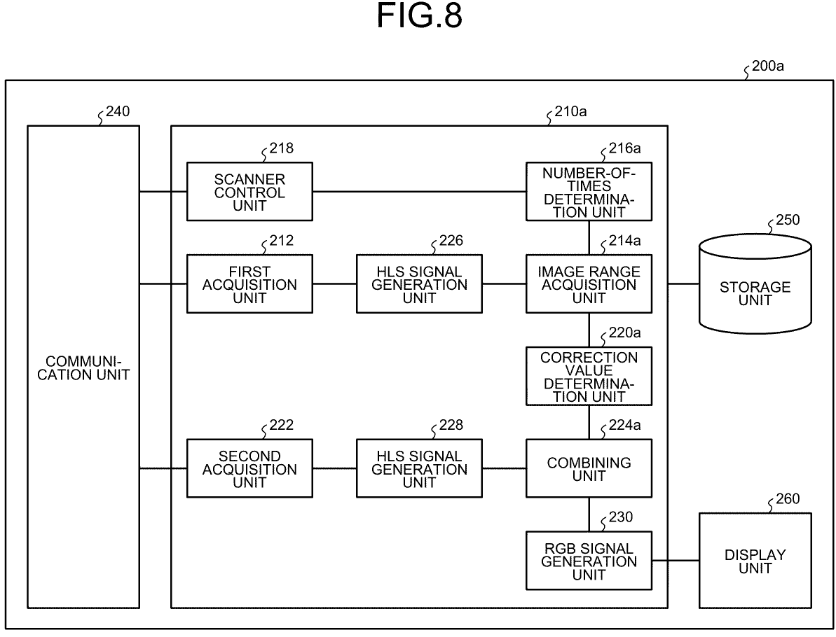

First, a detailed configuration of an image processing apparatus 200a according to the present embodiment is explained with reference to FIG. 8. FIG. 8 is a block diagram illustrating a configuration example of the image processing apparatus 200a according to the second embodiment of the present disclosure. Specifically, as illustrated in FIG. 8, the image processing apparatus 200a can mainly include a processing unit 210a, the communication unit 240, the storage unit 250, and the display unit 260. Functional blocks of the image processing apparatus 200a are sequentially explained below. However, since the functional blocks other than the processing unit 210a are common to the functional blocks of the image processing apparatus 200 according to the first embodiment, the explanation of the functional blocks other than the processing unit 210a is omitted and only the processing unit 210a is explained.

### Processing Unit 210a

As in the first embodiment, the processing unit 210a can control the DPI scanner 100 and process a digital image received from the DPI scanner 100 and is realized by, for example, a processing circuit such as a CPU. Specifically, as illustrated in FIG. 8, the processing unit 210a mainly includes the first acquisition unit (the first pixel signal acquisition unit) 212, an image range acquisition unit (a range acquisition unit) 214a, a number-of-times determination unit 216a, the scanner control unit (the control unit) 218, a correction value determination unit 220a, the second acquisition unit (the second pixel signal acquisition unit) 222, and a combining unit (an image generation unit) 224a. The processing unit 210a further includes HLS signal generation units 226 and 228 and an RGB signal generation unit 230. The functional units of the processing unit 210a are sequentially explained below. However, explanation is omitted about the functional units common to the first embodiment.

The image range acquisition unit 214a acquires a distribution width (a pixel value range) (a dynamic range) of the L values in primary image 400 converted into the HLS signal. Specifically, the image range acquisition unit 214 converts the L values of all the pixels included in the primary image 400 into, for example, a histogram indicating a distribution of frequencies of the levels and acquires a maximum value and a minimum value of the L values. The image range acquisition unit 214a outputs the acquired maximum value and minimum value to the number-of-times determination unit 216a and the correction value determination unit 220a explained below.

The number-of-times determination unit 216a determines the number of times of photographing N for the divided regions (the regions to be photographed) 500 in the biological tissue specimen based on the minimum value and the maximum value of the L values acquired by the image range acquisition unit 214a explained above and outputs the number of times of photographing N to the scanner control unit 218 explained below. Note that a specific method of determining the number of times of photographing N in the number-of-times determination unit 216a is explained below.

The correction value determination unit 220a determines the minimum value of the L values acquired by image range acquisition unit 214 as a correction value and outputs the correction value to combining unit 224a explained below.

The combining unit 224a can apply image processing to N secondary images (second photographed images) 402 of the divided regions 500 converted into an HLS signal received from the HLS signal generation unit 228 explained below. Specifically, the combining unit 224a can perform correction by subtracting the correction value (specifically, the minimum value of the L values) received from the correction value determination unit 220a explained above from the L value of the pixels of the N secondary images 402 of the divided regions 500 converted into the HLS signal. Then, the combining unit 224a adds up the corrected L values of the same pixels of the N secondary images 402 in the divided regions 500. Further, the combining unit 224a averages the H values and the S values of the same pixels of the N secondary images 402 in the divided regions 500. Then, the combining unit 224a outputs the obtained addition value or average value to the RGB signal generation unit 230 explained below. That is, the combining unit 224a can acquire the composite image 404 of the divided regions 500 represented by an HLS value. Then, in the present embodiment, the contrast in the composite image 404 can be further increased by adding up the L values. Noise in the composite image 404 can be further reduced by averaging the H values and the S values. Note that a specific method for the addition and the averaging by the combining unit 224a is explained below.

The HLS signal generation units 226 and 228 can convert the primary image 400 or the secondary image 402 of the biological tissue specimen acquired by the first acquisition unit 212 or the second acquisition unit 222 from an RGB signal into an HLS signal.

The RGB signal generation unit 230 can convert the HLS signal into an RGB signal using the added-up L value and the averaged H value and the averaged S value received from the combining unit 224a and acquire the composite image 404 of the divided regions 500.

### 3.2 Image Processing Method

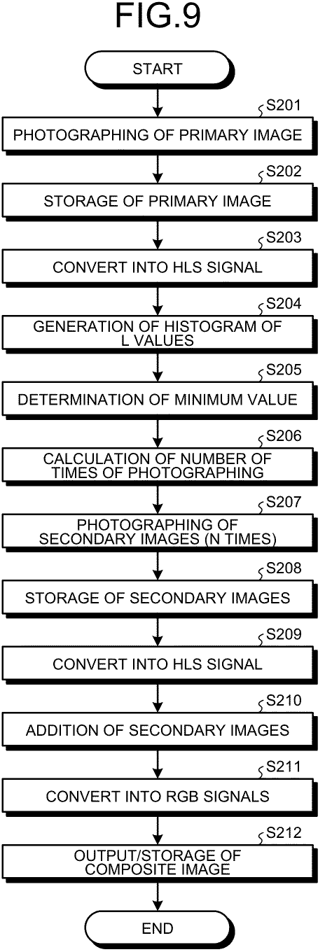

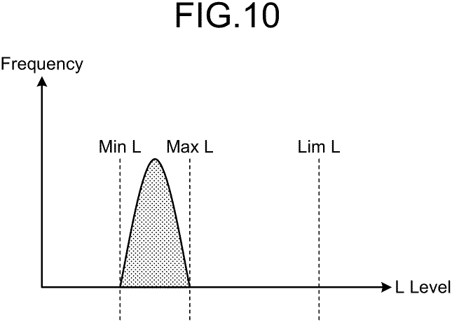

Subsequently, an image processing method according to the present embodiment is explained with reference to FIG. 9 and FIG. 10. FIG. 9 is a flowchart illustrating an example of image processing according to the present embodiment. FIG. 10 is an explanatory diagram for explaining the image processing according to the present embodiment. Specifically, as illustrated in FIG. 9, the image processing method according to the present embodiment can include steps from step S201 to step S212. Details of these steps according to the present embodiment are explained below. Note that, in the following explanation, only differences from the points in the first embodiment explained above are explained. Explanation is omitted about points common to the points in the first embodiment.

Since step S201 and step S202 are the same as step S101 and step S102 of the first embodiment illustrated in FIG. 6, explanation of the steps is omitted here.

The image processing system 10 converts the primary image 400 from an RGB signal into an HLS signal (step S203).

Then, the image processing system 10 generates a histogram of L values from the primary image 400 converted into the HLS signal (step S204). The generated histogram is illustrated in, for example, FIG. 10. In FIG. 10, a histogram indicating a distribution of the L values is illustrated. In the histogram, the L values are divided into predetermined ranges (levels) to indicate frequencies of the ranges.

Then, the image processing system 10 determines, as a correction value, a minimum value (Min L) (see FIG. 10) in the L values of all the pixels included in the primary image 400 (step S205).

Subsequently, the image processing system 10 calculates the number of times of photographing N (step S206). Specifically, in the subsequent processing, the L values of the same pixels of the N secondary images 402 of the divided regions 500 are added up. At that time, the number of times of photographing N is determined based on the following Formula (4) so that the added-up L value does not exceed an upper limit value (Lim L) (see FIG. 10) of the L values set in advance according to the characteristics of the sensor unit 104. In Formula (4), Max L is a maximum value of the L values of the histogram of the primary image 400 and Min L is a minimum value of the L values of the histogram of the primary image 400 (see FIG. 10).

$$N = L i m L / \left(M a x L - M i n L\right)$$

Since step S207 and step S208 are the same as step S106 and step S107 of the first embodiment illustrated in FIG. 6, explanation of the step is omitted here.

The image processing system 10 converts the N secondary images 402 of the divided regions 500 from an RGB signal into an HLS signal (step S209).

According to the following Formula (5), the image processing system 10 subtracts the minimum value (Min L) of the L values from the L values of the pixels of the N secondary images 402 of the divided regions 500 and adds up the L values after the subtraction for each of the same pixels. Further, the image processing system 10 averages the H values and the S values for each of the same pixels of the N secondary images 402 of the divided regions 500 according to the following Formula (5) (step S 210). In Formula (5), InputImageHi (x, y), InputImageSi (x, y), and InputImageLi (x, y) are H values, S values, and L values of the pixels of the secondary images 402 and OutImageH (x, y), OutImageS (x, y), and OutImageL (x, y) are average values of the H values and the S values and an add-up value of the L values for each of the same pixels of the N secondary images 402. In the present embodiment, the contrast in the composite image 404 can be further increased by adding up the L values and the noise in the composite image 404 can be further reduced by averaging the H values and the S values.

$$\begin{matrix}O u t I m a g e H \left(x , y\right) = {\sum }_{i = 0}^{N} \left(I n p u t I m a g e {H}_{i} \left(x , y\right)\right) / N \\ O u t I m a g e S \left(x , y\right) = {\sum }_{i = 0}^{N} \left(I n p u t I m a g e {S}_{i} \left(x , y\right)\right) / N \\ O u t I m a g e L \left(x , y\right) = {\sum }_{i = 0}^{N} \left(I n p u t I m a g e {L}_{i} \left(x , y\right) - M i n L\right)\end{matrix}$$

The image processing system 10 converts an HLS signal including the added-up L value and the averaged H value and S value obtained in step S210 explained above into an RGB signal and acquires the composite image 404 of the divided regions 500 (step S211).

Since step S212 is the same as step S109 of the first embodiment illustrated in FIG. 6, explanation of the step is omitted here.

As explained above, in the present embodiment, the contrast in the composite image 404 is further increased by adding up the L values and the noise in the composite image 404 can be further reduced by averaging the H values and the S values.

### 3.3 Modification

In the second embodiment described above, it explained that the combining unit 224a adds up the L values of the same pixels of the N secondary images 402 of the divided regions 500 and averages the H values and the S values. However, the present embodiment is not limited to this. In a modification of the present embodiment, for example, the combining unit 224a may add up, instead of the L values, the S values of the same pixels of the N secondary images 402 of the divided regions 500 and average the L values and the H values. In this case, the number of times of photographing N may be determined or the correction value may be determined using a minimum value and a maximum value of the S values in the primary image 400. Furthermore, in this case, the number of times of photographing N may be determined or the correction value may be determined using minimum values and maximum values of the L values and the S values in the primary image 400. Alternatively, in the modification of the present embodiment, the L values and the S values may be added up.

Further, the embodiment explained above is not limited to the use of the HLS color space. A YCC color space for expressing a color image with luminance (Y), a blue hue and chroma (Cb), and a red hue and chroma (Cr) may be used.

### 4. Third Embodiment

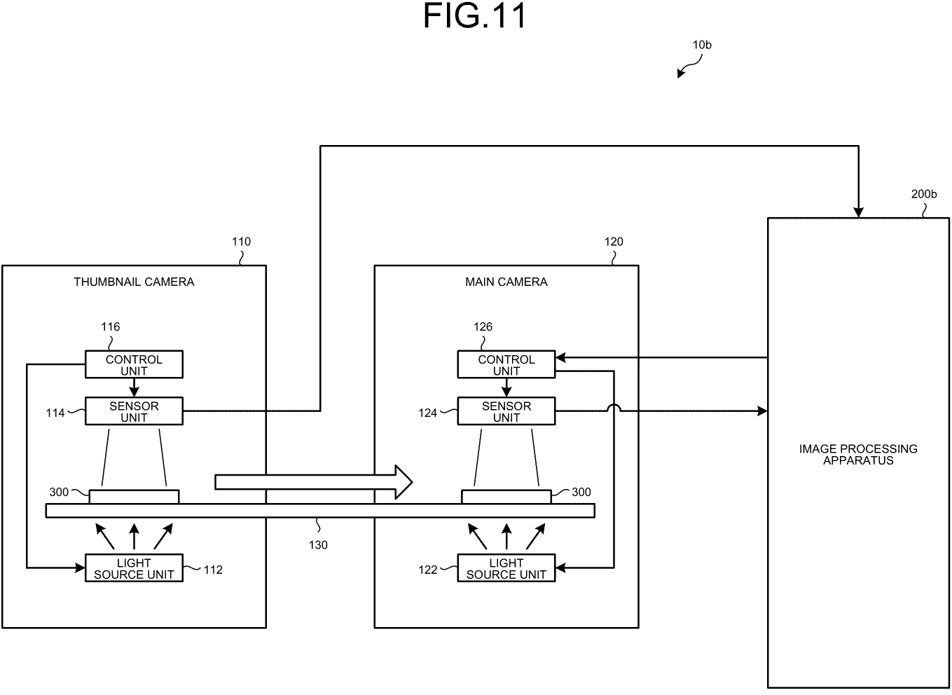

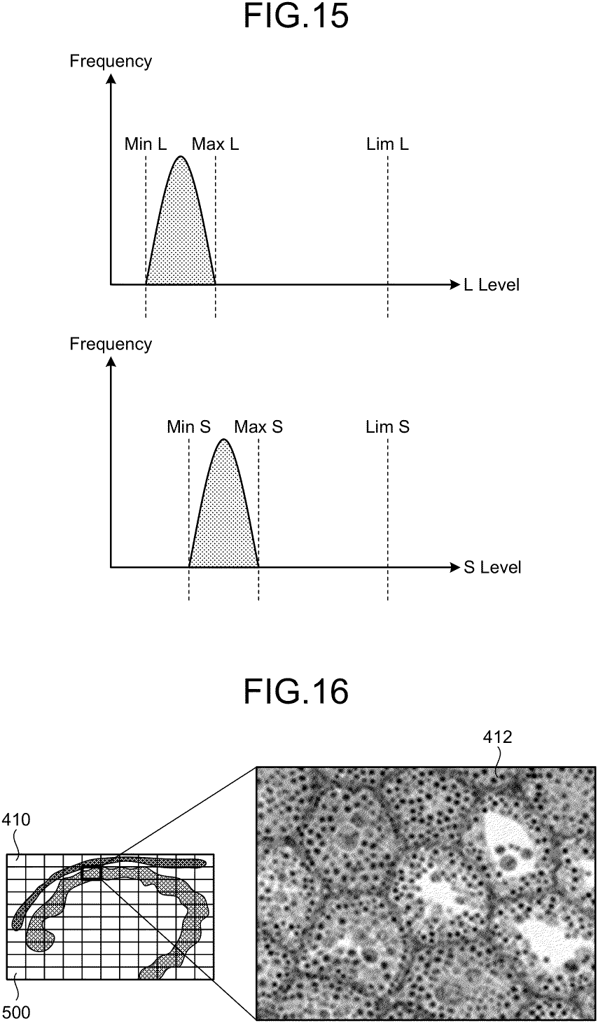

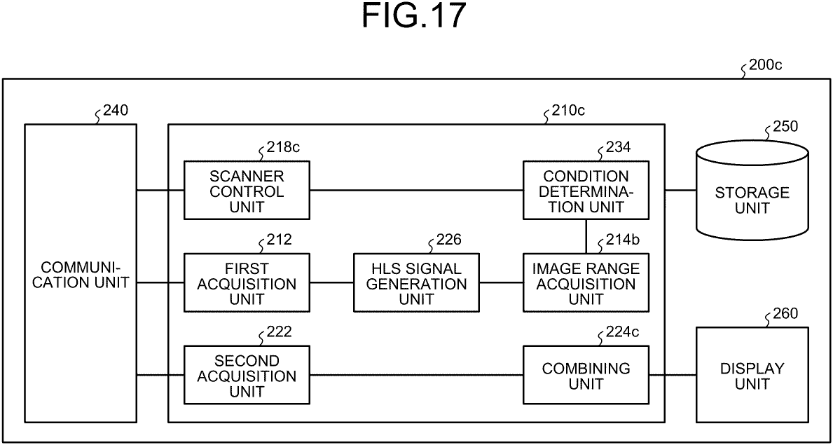

Incidentally, in a photographing apparatus that photographs a high magnification image of a biological tissue specimen using a digital imaging system, in some case, a high magnification image of the entire biological tissue specimen is acquired by photographing a high magnification image for each of partial regions (for example, the divided regions 500) of the biological tissue specimen according to frame allocation beforehand and joining (stitching) a plurality of obtained images. At this time, in order to determine the frame allocation, for example, a thumbnail image having low resolution, which is an image of the entire biological tissue specimen, is acquired by a thumbnail camera 110 (see FIG. 11) explained below and virtual division (frame allocation) of the biological tissue specimen is determined based on the thumbnail image. Therefore, in the present embodiment, such a thumbnail image is used as the primary image 410 (see FIG. 16). By using such a thumbnail image as the primary image 410, a processing time related to the image processing can be reduced. Details of the present embodiment are explained below.

### 4.1 Image Processing System

First, a configuration example of an image processing system 10b according to a third embodiment of the present disclosure is explained with reference to FIG. 11. FIG. 11 is a block diagram illustrating a configuration example of an image processing system 10b according to a third embodiment of the present disclosure. As illustrated in FIG. 11, the image processing system 10a according to the present embodiment can include a thumbnail camera 110, a main camera 120, and an image processing apparatus 200b. The thumbnail camera 110, the main camera 120, and the image processing apparatus 200b may be communicably connected to one another via various wired or wireless communication networks. In the present embodiment, a slide loader 130 that conveys the slide 300 from the thumbnail camera 110 to the main camera 120 is provided between the thumbnail camera 110 and the main camera 120. For example, the slide loader 130 can convey the slide 300 from the thumbnail camera 110 to the main camera 120 by being controlled by the image processing apparatus 200b. By providing such a slide loader 130, the slide 300 is automatically conveyed from the thumbnail camera 110 to the main camera 120. An overview of devices included in the image processing system 10b according to the present embodiment is explained.

### Thumbnail Camera 110

The thumbnail camera 110 is a digital camera that photographs an entire image of a biological tissue specimen and has a form including a light source unit 112, a sensor unit 114, a control unit 116, and the slide loader 130 functioning as a stage like the DPI scanner 100 explained above. Specifically, the control unit 116 controls the light source unit 112 and the sensor unit 114 to photograph the entire image of the biological tissue specimen. A digital image obtained by the photographing is output to the image processing apparatus 200b as the primary image 410 (see FIG. 16).

Further, the thumbnail camera 110 may be provided with a mechanism for photographing incidental information (identification information of the biological tissue specimen, attribute information of the biological tissue specimen (region, age, sex, disease, and the like) of an organism), preparation condition information of the biological tissue specimen (a staining reagent and staining conditions), and the like) described on a label (not illustrated) stuck to the slide 300. In this case, a photographed digital image of the label may be output to the image processing apparatus 200b. The image processing apparatus 200b may directly acquire the incidental information using the digital image or may acquire the incidental information from an external server (not illustrated).

### Main Camera 120

The main camera 120 is, for example, a digital camera that photographs the divided regions 500 of the slide 300 of the biological tissue specimen at a larger magnification than that of the thumbnail camera 110 and has a form including a light source unit 122, a sensor unit 124, a control unit 126, and the slide loader 130 functioning as a stage like the DPI scanner 100 explained above. Specifically, the control unit 126 controls the light source unit 112 and the sensor unit 114 according to a command from the image processing apparatus 200b to photograph the divided regions 500 of the biological tissue specimen. A digital image obtained by the photographing is output to the image processing apparatus 200b as the secondary image 412 (see FIG. 16).

Note that, in the above explanation, it is explained that the primary image (the photographed image related to the first pixel signal) 410 photographed by the thumbnail camera 110 has a wider angle of view than the secondary image (the photographed image related to the second pixel signal) 412 photographed by the main camera 120. However, the present embodiment is not limited to this. For example, the angles of view may be the same. In the present embodiment, the resolution of the primary image 410 photographed by the thumbnail camera 110 may be lower than or may be the same as the resolution of the secondary image 412 photographed by the main camera 120.

### Image Processing Apparatus 200 B

The image processing apparatus 200b is an apparatus having a function of controlling the main camera 120 based on the primary image 410 photographed by the thumbnail camera 110 and processing a plurality of secondary images 412 photographed by the main camera 120.

### 4.2 Image Processing Apparatus

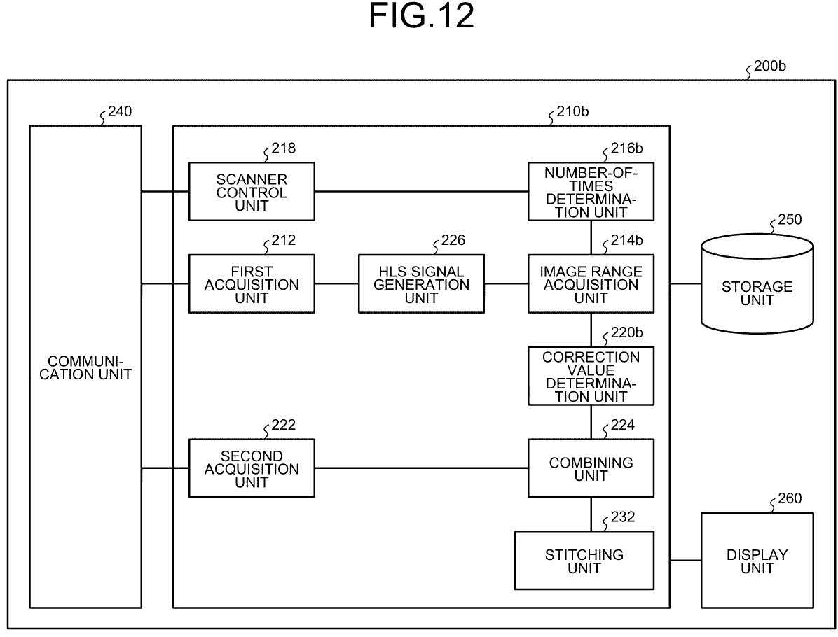

Subsequently, a detailed configuration of the image processing apparatus 200b according to the present embodiment is explained with reference to FIG. 12. FIG. 12 is a block diagram illustrating a configuration example of the image processing apparatus 200b according to the third embodiment of the present disclosure. Specifically, as illustrated in FIG. 12, the image processing apparatus 200b can mainly include a processing unit 210b, the communication unit 240, the storage unit 250, and the display unit 260. Functional blocks of the image processing apparatus 200b are sequentially explained below. However, since the functional blocks other than the processing unit 210b are common to the functional blocks of the image processing apparatuses 200 and 200a according to the first and second embodiments, explanation of the functional blocks other than the processing unit 210b is omitted. Only the processing unit 210b is explained.

### Processing Unit 210b

The processing unit 210b can control the main camera 120 based on the primary image 410, which is the entire image of the biological tissue specimen, received from the thumbnail camera 110 and process the plurality of secondary images 412 of the divided regions 500 of the biological tissue specimen received from the main camera 120. The processing unit 210b is realized by, for example, a processing circuit such as a CPU. Specifically, as illustrated in FIG. 8, the processing unit 210b mainly includes the first acquisition unit (the first pixel signal acquisition unit) 212, an image range acquisition unit (a range acquisition unit) 214b, a number-of-times determination unit 216b, the scanner control unit (the control unit) 218, a correction value determination unit 220b, the second acquisition unit (the second pixel signal acquisition unit) 222, the combining unit (the image generation unit) 224, and the HLS signal generation unit 226. Further, the processing unit 210b includes a stitching unit 232. The functional unit of the processing unit 210b are sequentially explained below. However, explanation is omitted about of the functional units common to the first and second embodiments.

The image range acquisition unit 214b acquires a distribution width (a dynamic range) of L values and S values in primary image 410 converted into an HLS signal. Specifically, the image range acquisition unit 214 converts the L value and the S value of each pixel included in the primary image 410 into, for example, histograms indicating a frequency distribution of each level (obtained by dividing the pixel value for each predetermined value range), and acquires the maximum values and the minimum values of the L values and the S values. The image range acquisition unit 214b outputs the acquired maximum values and the acquired minimum values to the number-of-times determination unit 216b and the correction value determination unit 220b explained below.

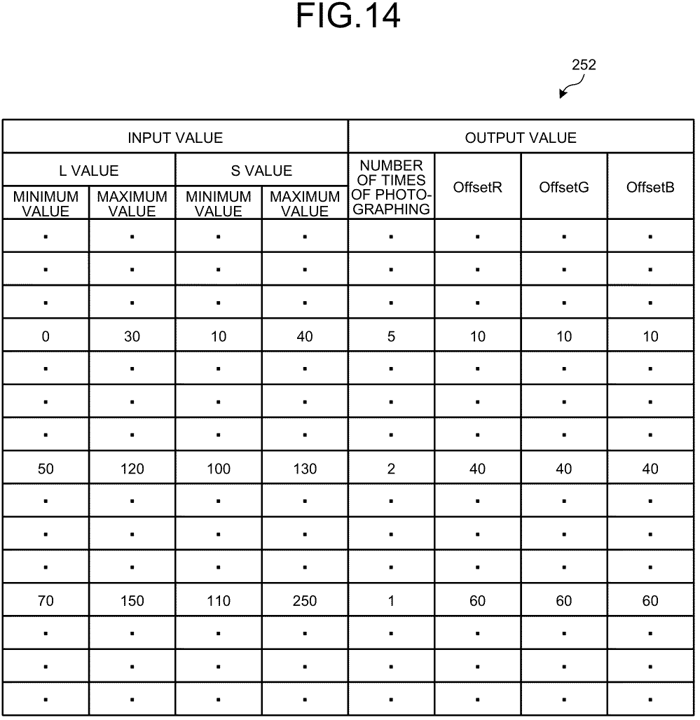

The number-of-times determination unit 216b refers to a table (a predetermined table) 252 (see FIG. 14), determines the number of times of photographing N for the same divided regions (regions to be photographed) 500 in the biological tissue specimen based on the minimum values and the maximum values of the L values and the S values acquired by the image range acquisition unit 214a, and outputs the number of times of photographing N to the scanner control unit 218.

The correction value determination unit 220b refers to the table (the predetermined table) 252 (see FIG. 14), determines an offset value (Offset) serving as a correction value based on the minimum values and the maximum values of the L values and the S values acquired by image range acquisition unit 214 explained above, and outputs the offset value to the combining unit 224 explained below.

In the present embodiment, since the number of times of photographing N and the correction value are determined using both of the L values and the S values, it is possible to obtain a final image (not illustrated) in which a balance between the L values and the S values is considered.

Note that the table 252 illustrated in FIG. 14 stores, for example, the number of times of photographing N and an offset value optimum for obtaining a clear final image for each index (for example, the maximum values and the minimum values of the L values and the S values) obtained experimentally based on photographing in the past. Note that, in the present embodiment, the table 252 may be generated in advance based on a photographed image quality model obtained by performing machine learning of a photographing history in the past (photographing conditions, quality of a composite image, and the like). The values illustrated in FIG. 14 are only examples. Values stored in the table 252 according to the present embodiment are not limited to the values illustrated in FIG. 14.

In the above explanation, it is explained that the number-of-times determination unit 216b and the correction value determination unit 220b select the number of times of photographing N and the offset value linked to the minimum values and the maximum values of the L values and the S values from the table 252. However, the present embodiment is not limited to this. In the present embodiment, for example, the number-of-times determination unit 216b and the correction value determination unit 220b may select a table to be used based on supplementary information (imparted information) described in a label (not illustrated) stuck to the slide 300 or may select the number of times of photographing N and the offset value from the table 252.

The stitching unit 232 joins tertiary images (not illustrated) related to the divided regions 500 different from one another obtained by adding up the N secondary images 412 in the combining unit 224 according to a positional relationship among the divided regions 500 to generate a final image (not illustrated).

### 4.3 Image Processing Method

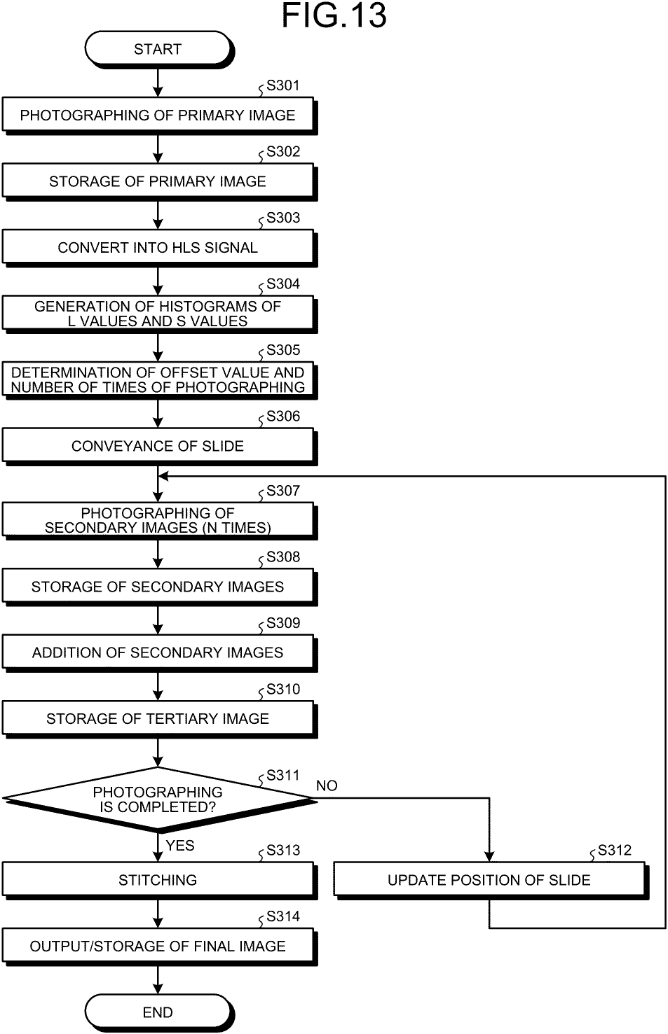

Subsequently, an image processing method according to the present embodiment is explained with reference to FIG. 13 to FIG. 16. FIG. 13 is a flowchart illustrating an example of image processing according to the present embodiment. FIG. 14 is an explanatory diagram for explaining an example of the table 252 according to the present embodiment. FIG. 15 and FIG. 16 are explanatory diagrams for explaining image processing according to the present embodiment. Specifically, as illustrated in FIG. 13, the image processing method according to the present embodiment can include steps from step S301 to step S314. Details of these steps according to the present embodiment are explained below. Note that, in the following explanation, only differences from the first and second embodiments are explained. Explanation is omitted about points common to the first and second embodiments.

First, the image processing system 10 b photographs the primary image 410 (see FIG. 16), which is the entire image of the biological tissue specimen, with the thumbnail camera 110 (step S101).

Since step S302 and step S303 are the same as steps S202 and S203 in the second embodiment illustrated in FIG. 9, explanation of the steps is omitted here.

Subsequently, the image processing system 10b generates histograms of L values and S values from the primary image 410 (see FIG. 16) converted into the HLS signal (step S304). The generated histograms are illustrated in, for example, FIG. 15. In FIG. 15, histograms showing distributions of the L values and the S values are illustrated. In the histograms, the L values and the S values are divided into predetermined ranges (levels) to indicate frequencies of the ranges.

The image processing system 10b selects the number of times of photographing N and offset values (Offset R, Offset G, Offset B) linked to the minimum values and the maximum values of the L values and the S values from the table 252 illustrated in FIG. 14 and determines the number of times of photographing N and the offset values (step S305). In the present embodiment, since the number of times of photographing N and the offset values are determined using the minimum values and the maximum values of both of the L values and the S values, it is possible to obtain a final image (not illustrated) in which a balance between the L values and the S values is considered. Then, the image processing system 10b conveys the slide 300 from the thumbnail camera 110 to the main camera 120 (step S306).

The image processing system 10b photographs the secondary image 412 of one divided region 500 of the biological tissue specimen by the number of times of photographing N with the main camera 120 (step S307).

Since step S308 is the same as step S208 in the second embodiment illustrated in FIG. 9, explanation of the step is omitted here.

According to the following Formula (6), the image processing system 10b subtracts the offset values (Offset R, Offset G, Offset B) from the RGB values of the pixels of the N secondary images 402 and adds up the RGB values after the subtraction for each of the same pixels to thereby synthesize tertiary images (not illustrated) (step S309).

$$\begin{matrix}O u t I m a g e R \left(x , y\right) = {\sum }_{i = 0}^{N} \left(I n p u t I m a g e {R}_{i} \left(x , y\right) - O f f s e t R\right) \\ O u t I m a g e G \left(x , y\right) = {\sum }_{i = 0}^{N} \left(I n p u t I m a g e {G}_{i} \left(x , y\right) - O f f s e t G\right) \\ O u t I m a g e B \left(x , y\right) = {\sum }_{i = 0}^{N} \left(I n p u t I m a g e {B}_{i} \left(x , y\right) - O f f s e t B\right)\end{matrix}$$

Subsequently, the image processing system 10b stores the tertiary images (not illustrated) of the colors in a tertiary image accumulation unit (not illustrated) of the storage unit 250 (step S310).

Then, the image processing system 10b determines whether N times of photographing of the secondary images 412 is completed for all the divided regions 500 (step S311). The image processing system 10b proceeds to step S313 when the photographing is completed (step S311: Yes) and proceeds to step S312 when the photographing is not completed (step S105: No).

Subsequently, the image processing system 10b updates a photographing position of the slide 300 in order to photograph the secondary image 412 of the divided region 500 to be the next photographing target and returns to step S307 explained above (step S312). That is, in the present embodiment, update of the photographing position of the slide 300, the N times of photographing of the secondary image 412, and the synthesis of the tertiary image (not illustrated) are repeated until the N times of photographing of the secondary image 412 is completed for all the divided regions 500.

Then, the image processing system 10b generates a final image (not illustrated) by joining (stitching) a plurality of tertiary images (not illustrated) related to the divided regions 500 different from one another according to a positional relation among the divided regions 500 (step S313). Further, the image processing system 10b outputs the final image to the display unit 260 and stores the final image in a composite image accumulation unit (not illustrated) of the storage unit 250 (step S314).

As explained above, according to the present embodiment, since the number of times of photographing N and the offset values are determined using the minimum values and the maximum values of both the L values and the S values, it is possible to obtain a final image (not illustrated) in which a balance between the L values and the S values is considered. Further, according to the present embodiment, by using the thumbnail image as the primary image 410, a processing time related to the image processing can be reduced.

In the embodiment explained above, the present disclosure is not limited to the use of the HLS color space. A YCC color space in which a color image is expressed by luminance, a blue hue and chroma and a red hue and chroma may be used.

### 5. Fourth Embodiment

In a fourth embodiment explained below, a light amount of light with which a biological tissue specimen is irradiated is adjusted by the light source unit 102 of the DPI scanner 100 according to an analysis result of the primary image 400 and a plurality of secondary images 402 are acquired. In the present embodiment, a clear composite image 404 can be acquired by adding up the N secondary images 402 photographed under the condition that the light amount is suitably adjusted. Further, according to the present embodiment, color floating and black floating in the composite image 404 can be suppressed by suitably adjusting the light amount. Details of such an embodiment are explained below.

Note that configuration examples of the image processing system 10 and the DPI scanner 100 according to the fourth embodiment are common to the image processing system 10 and the DPI scanner 100 according to the first embodiment. Therefore, the explanation of the configurations of the image processing system 10 and the DPI scanner 100 according to the first embodiment and FIG. 3 referred to in the explanation can be referred to. Therefore, here, explanation of the image processing system 10 and the DPI scanner 100 according to the present embodiment is omitted.

### 5.1 Image Processing Apparatus

First, a detailed configuration of an image processing apparatus 200c according to the present embodiment is explained with reference to FIG. 17. FIG. 17 is a block diagram illustrating a configuration example of the image processing apparatus 200c according to the fourth embodiment of the present disclosure. Specifically, as illustrated in FIG. 17, the image processing apparatus 200c can mainly include a processing unit 210c, the communication unit 240, the storage unit 250, and the display unit 260. Functional blocks of the image processing apparatus 200c are sequentially explained below. However, since the functional blocks other than the processing unit 210c are common to the functional blocks of the image processing apparatuses 200, 200a, and 200b according to the first to third embodiments, explanation of the functional blocks other than the processing unit 210c is omitted. Only the processing unit 210c is explained.

### Processing Unit 210c

As in the first embodiment, the processing unit 210c can control the DPI scanner 100 and process a digital image received from the DPI scanner 100 and is realized by, for example, a processing circuit such as a CPU. Specifically, as illustrated in FIG. 17, the processing unit 210c mainly includes the first acquisition unit (the first pixel signal acquisition unit) 212, the image range acquisition unit (the range acquisition unit) 214b, a scanner control unit (a control unit) 218c, the second acquisition unit (the second pixel signal acquisition unit) 222, a combining unit (an image generation unit) 224c, and an HLS signal generation unit 226. Further, the processing unit 210c further includes a condition determination unit 234. The functional units of the processing unit 210c are sequentially explained below. However, explanation of the functional units common to the first to third embodiments is omitted.

The scanner control unit 218c generates, based on the number of times of photographing N and a light amount determined by the condition determination unit 234 explained below, a command for controlling the DPI scanner 100 and controls the DPI scanner 100 via the communication unit 240.

The combining unit 224c superimposes (adds up) the N secondary images 402 of the divided regions 500 received from the second acquisition unit 222 to generate the composite image 404. For example, the combining unit 224 can obtain the composite image 404 for each of colors by simply adding up RGB values of the same pixels of the N secondary images 402.

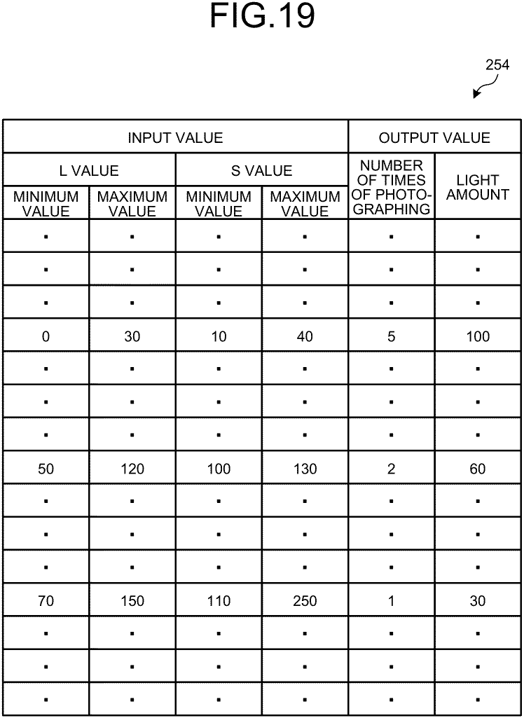

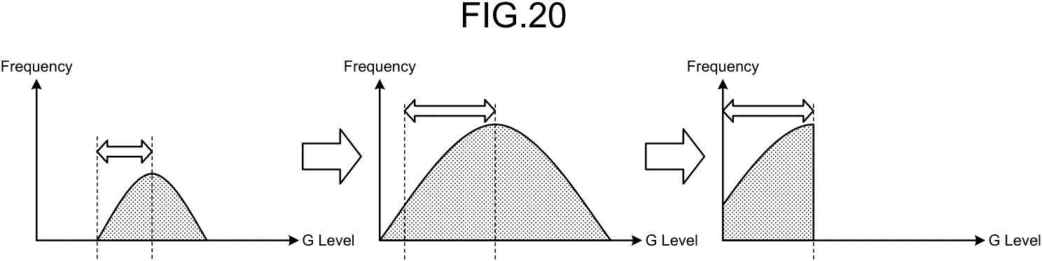

The condition determination unit 234 refers to the table 254 (see FIG. 19), determines the number of times of photographing N and a light amount for at least a part of the divided regions (the regions to be photographed) 500 in the biological tissue specimen based on the minimum values and the maximum values of the L values and the S values acquired by the image range acquisition unit 214b and outputs the number of times of photographing N and the light amount (which may be irradiation intensity or an irradiation time corresponding to the light amount) to the scanner control unit 218c. Note that, in the present embodiment, the condition determination unit 234 is not limited to determining the number of times of photographing N and the light amount and may also determine a wavelength or the like of the irradiation light.

Note that the table 254 illustrated in FIG. 19 stores, for example, the number of times of photographing N and a light amount optimum for obtaining a clear composite image 404 for each of indexes (for example, the maximum values and the minimum values of the L values and the S values) obtained experimentally based on photographing in the past. Note that, in the present embodiment, the table 254 may be generated in advance based on a photographed image quality model obtained by performing machine learning of a photographing history in the past (photographing conditions, quality of the composite image 404, and the like). The values illustrated in FIG. 19 are only examples. Values stored in the table 254 according to the present embodiment are not limited to the values illustrated in FIG. 19.

### 5.2 Image Processing Method

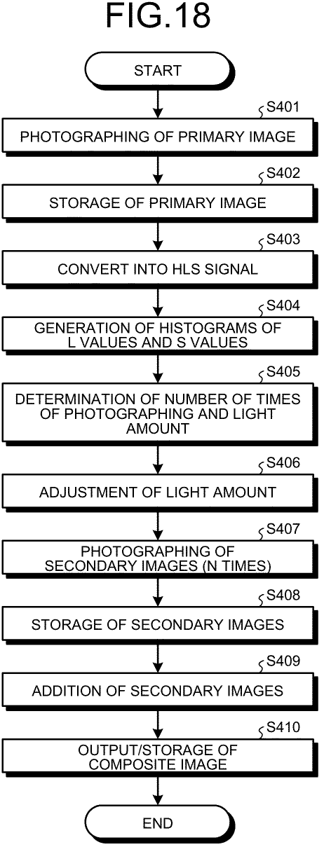

Subsequently, an image processing method according to the present embodiment is explained with reference to FIG. 18 and FIG. 19. FIG. 18 is a flowchart illustrating an example of image processing according to the present embodiment. FIG. 19 is an explanatory diagram for explaining an example of the table 254 according to the present embodiment. Specifically, as illustrated in FIG. 18, the image processing method according to the present embodiment can include steps from step S401 to step S410. Details of these steps according to the present embodiment are explained below. In the following explanation, only differences from the first to third embodiments are explained. Explanation is omitted about points common to the first to third embodiments.

Since step S401 to step S403 are the same as step S201 to step S203 in the second embodiment illustrated in FIG. 9. Therefore, explanation of the steps is omitted here.

Since step S404 is the same as step S304 in the third embodiment illustrated in FIG. 13, explanation of the step is omitted here.

The image processing system 10 selects the number of times of photographing N and the light amount linked to the minimum values and the maximum values of the L values and the S values from the table 254 illustrated in FIG. 19 and determines the number of times of photographing N and the light amount (step S405). The image processing system 10 adjusts a light amount (specifically, irradiation intensity, an irradiation time, and the like) of the light source unit 102 of the DPI scanner 100 according to the determined light amount (step S406).

Since step S407 and step S408 are the same as step S207 and step S208 of the second embodiment illustrated in FIG. 9, explanation of the steps is omitted here.

The image processing system 10 synthesizes the composite image 404 of the colors by adding up RGB values of the same pixels of the N secondary images 402 according to the following Formula (7) (step S409).

$$\begin{matrix}O u t I m a g e R \left(x , y\right) = {\sum }_{i = 0}^{N} \left(I n p u t I m a g e {R}_{i} \left(x , y\right)\right) \\ O u t I m a g e G \left(x , y\right) = {\sum }_{i = 0}^{N} \left(I n p u t I m a g e {G}_{i} \left(x , y\right)\right) \\ O u t I m a g e B \left(x , y\right) = {\sum }_{i = 0}^{N} \left(I n p u t I m a g e {B}_{i} \left(x , y\right)\right)\end{matrix}$$

Since step S410 is the same as step S212 in the second embodiment illustrated in FIG. 9, explanation of the step is omitted here.

As explained above, according to the present embodiment, a clear composite image 404 can be acquired by adding up the N secondary images 402 photographed under a condition that a light amount is suitably adjusted according to an analysis result of the primary image 400. Further, according to the present embodiment, color floating and black floating in the composite image 404 can be suppressed by suitably adjusting the light amount.

### 6. Fifth Embodiment

Incidentally, for example, when a biological tissue specimen is stained using silver mesenamine periodate (PAM) or periodic acid Schiff (PAS) staining, a predetermined tissue is stained more deeply. That is, by staining the biological tissue specimen using a specific staining reagent, the predetermined tissue clearly emerges and analysis can be performed focusing on the tissue. When such a tissue of attention is limited, in order to more accurately perform the analysis, it is preferable to acquire an image in which the contrast of the stained tissue of attention is further enhanced. Therefore, in the present embodiment, by determining the number of times of photographing N and a correction value according to a type of a staining reagent and a dynamic range of RGB values in a range of a tissue of attention in the primary image 400, it is possible to acquire an image in which the details of the stained tissue of attention are clearly seen and contrast is further enhanced.

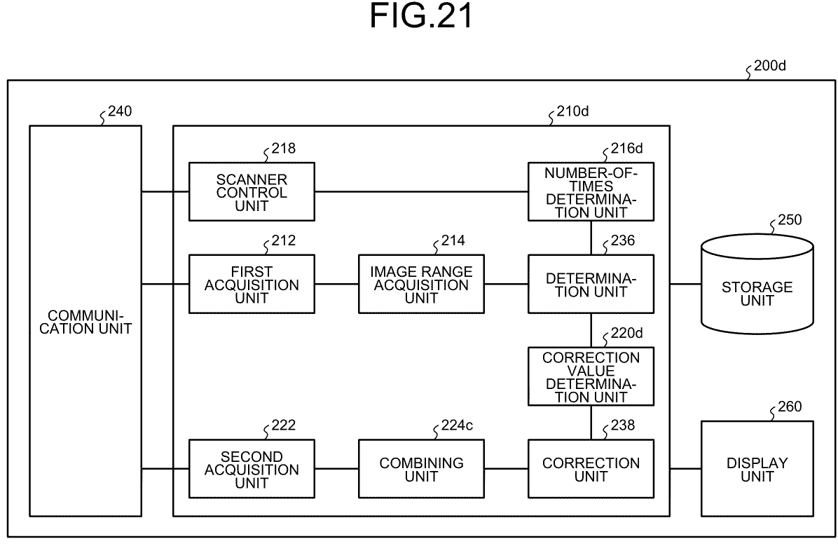

A concept of the present embodiment is explained with reference to FIG. 20. FIG. 20 is an explanatory diagram for explaining the present embodiment and, more specifically, illustrates, from the left to the right, a distribution of G values in the secondary image 402, a distribution of G values in the case in which the N secondary images 402 are added up, and a distribution of G values in an image after correction in the present embodiment. In the present embodiment, since a distribution width (a dynamic range) of pixel values corresponding to the tissue of attention in an image after correction is suitably expanded, details of the tissue of attention are clear and the tissue of attention can be more easily seen.

Specifically, when G values are explained as an example, first, in the present embodiment, as in the embodiments explained above, a biological tissue specimen stained with a staining reagent A is photographed N times to acquire N secondary images 402. At this time, a distribution of the G values of the secondary images 402 is the left figure in FIG. 20. Then, also in the present embodiment as well, as in the embodiments explained above, by adding up the G values of the N secondary images 402, the range of the distribution of the G values is expanded as illustrated in the center figure of FIG. 20 and an added-up image (not illustrated) having a wide dynamic range can be obtained. If the display unit 260 is a wide dynamic range display device that can express the G values with a wide gradation width, even if the added-up image is displayed as it is, details of a region of attention can be clearly displayed. However, when the display unit 260 is a narrow dynamic range display device that can express the G values only with a limited narrow gradation width, it is sometimes difficult to clearly display details of the region of attention when the added-up image is displayed. Therefore, in the present embodiment, when there is a limitation in a range of the G values that can be displayed in this way, as illustrated in the right figure of FIG. 20, by cutting out the distribution width of the G values of the region corresponding to the tissue of attention by the correction, the tissue of attention can be displayed in a high contrast state. Details of such a present embodiment are explained below.

Note that, in the present embodiment, the biological tissue specimen is assumed to be a biological tissue specimen stained with one or a plurality of staining reagents.

Further, since the configuration examples of the image processing system 10 and the DPI scanner 100 according to the fifth embodiment are common to the image processing system 10 and the DPI scanner 100 according to the first embodiment, the explanation of the configurations of the image processing system 10 and the DPI scanner 100 according to the first embodiment and FIG. 3 referred to in the explanation can be referred to. Therefore, here, explanation of the image processing system 10 and the DPI scanner 100 according to the present embodiment is omitted.

### 6.1 Image Processing Apparatus

First, a detailed configuration of an image processing apparatus 200d according to the present embodiment is explained with reference to FIG. 21. FIG. 21 is a block diagram illustrating a configuration example of the image processing apparatus 200d according to a fifth embodiment of the present disclosure. Specifically, as illustrated in FIG. 21, the image processing apparatus 200d can mainly include a processing unit 210d, the communication unit 240, the storage unit 250, and the display unit 260. Functional blocks of the image processing apparatus 200d are sequentially explained. However, since the functional blocks other than the processing unit 210d are common to the functional blocks of the image processing apparatus 200 according to the first embodiment, explanation of the functional blocks other than the processing unit 210d is omitted. Only the processing unit 210d is explained.

### Processing Unit 210d

As in the first embodiment, the processing unit 210d can control the DPI scanner 100 and process a digital image received from the DPI scanner 100 and is realized by, for example, a processing circuit such as a CPU. Specifically, as illustrated in FIG. 21, the processing unit 210d mainly includes the first acquisition unit (the first pixel signal acquisition unit) 212, the image range acquisition unit (the range acquisition unit) 214, a the number-of-times determination unit 216d, the scanner control unit (the control unit) 218, a correction value determination unit 220d, the second acquisition unit (the second pixel signal acquisition unit) 222, and the combining unit (the image generation unit) 224c. Further, the processing unit 210d includes a determination unit (a specifying unit) 236 and a correction unit 238. The functional units of the processing unit 210d are sequentially explained. However, explanation is omitted about the functional units common to the first to fourth embodiments.

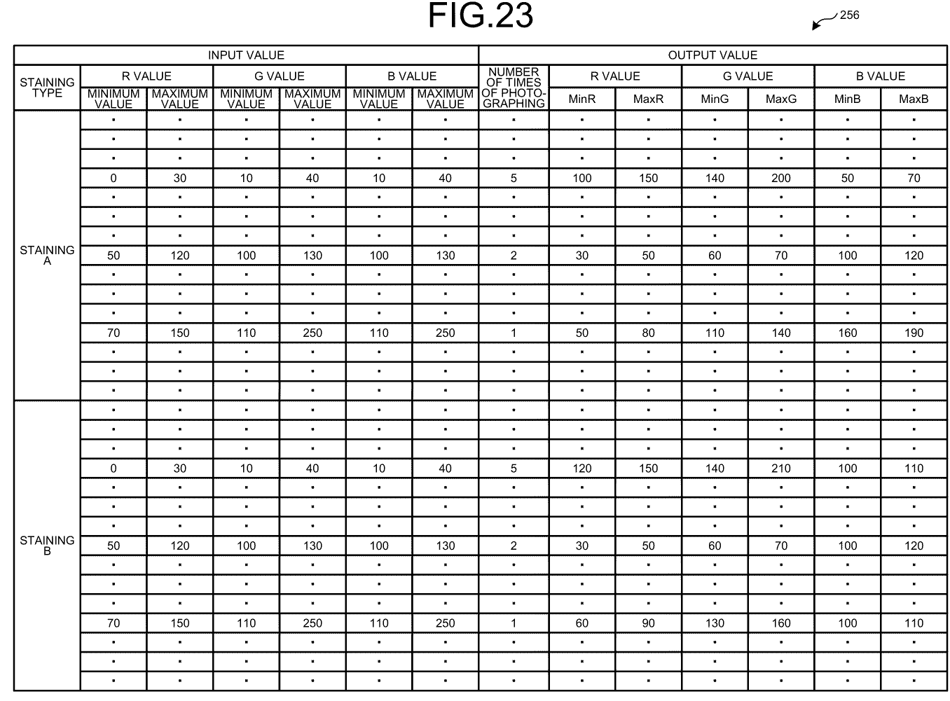

The number-of-times determination unit 216d refers to a table (a predetermined table) 256 (see FIG. 23), determines the number of times of photographing N for the divided regions (the regions to be photographed) 500 in the biological tissue specimen based on a type of a staining reagent for the biological tissue specimen determined by the determination unit 236 explained below and minimum values and maximum values of RGB values acquired by the image range acquisition unit 214, and outputs the number of times of photographing N to the scanner control unit 218.

The correction value determination unit 220d refers to the table (the predetermined table) 256 (see FIG. 23), determines a limited range (Min and Max) (see FIG. 23) of the RGB values based on the type of the staining reagent for the biological tissue specimen determined by the determination unit 236 explained below and the minimums value and the maximum values of the RGB values acquired by the image range acquisition unit 214, and outputs the limited range to the correction unit 238 explained below. That is, the limited range corresponds to the distribution width of the pixel values (specifically, the RGB values) of the region corresponding to the tissue of attention to be cut out explained with reference to FIG. 20.

Note that the table 256 illustrated in FIG. 23 stores, for example, an optimum number of times of photographing N and an optimum limited range of the RGB values for each of indexes (for example, the type of the staining reagent and the maximum values and the minimum values of the RGB values) experimentally obtained based on photographing in the past. Note that, in the present embodiment, the table 256 may be generated in advance based on a photographed image quality model obtained by performing machine learning of a photographing history in the past (photographing conditions, quality of a composite image, and the like). In addition, the values illustrated in FIG. 23 are only examples. Values stored in the table 256 according to the present embodiment are not limited to the values illustrated in FIG. 23.

The determination unit 236 determines (specifies) a type of the staining reagent for the biological tissue specimen based on shapes of histograms of the RGB values acquired by the image range acquisition unit 214 and outputs a determination result to the number-of-times determination unit 216d and the correction value determination unit 220d. In the present embodiment, for example, the type of the staining reagent may be determined based on a stained specimen recognition model obtained by machine learning. Specifically, for example, images of the biological tissue specimen stained by staining reagents are subjected to machine learning in advance. Feature points and feature amounts of histograms (pixel value ranges) of the RGB values in the images of the biological tissue specimen stained by the staining reagents are extracted to generate a staining reagent recognition model. Then, the determination unit 236 extracts, from the staining reagent recognition model, histograms of the RGB values same as or similar to feature points and feature amounts of the histograms of the RGB values acquired by the image range acquisition unit 214 and recognizes that a staining reagent linked to the extracted histograms is the staining reagent used in the biological tissue specimen. Note that, in the present embodiment, the type of the staining reagent for the biological tissue specimen is not limited to be determined based on the histograms. For example, the type of the staining reagent for the biological tissue specimen may be acquired by a manual input by the user.

The correction unit 238 can perform correction by cutting out the RGB values in a suitable range, that is, by limiting the range of the RGB values of the composite image 404 for each of the colors based on the limited range (Min and Max) of the RGB values determined by the correction value determination unit 220d (see FIG. 23).

### 6.2 Image Processing Method

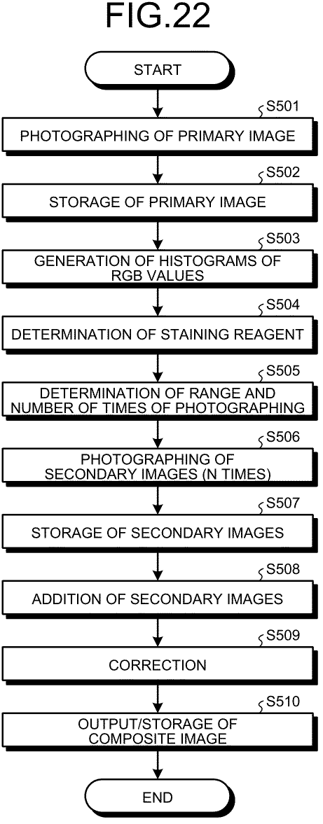

Subsequently, an information processing method according to the present embodiment is explained with reference to FIG. 22 and FIG. 23. FIG. 22 is a flowchart illustrating an example of image processing according to the present embodiment. FIG. 23 is an explanatory diagram for explaining an example of the table 256 according to the present embodiment. Specifically, as illustrated in FIG. 22, the image processing method according to the present embodiment can include steps from step S501 to step S510. Details of these steps according to the present embodiment are explained below. In the following explanation, only differences from the first to fourth embodiments are explained. Explanation is omitted about points common to the first to fourth embodiments.

Since step S501 to step S503 are the same as step S101 to step S103 in the first embodiment illustrated in FIG. 6, explanation of the steps is omitted here.

The image processing system 10 determines a type of a staining reagent of the biological tissue specimen based on shapes of the histograms of the RGB values (step S504) .

The image processing system 10 refers to the table 256 illustrated in FIG. 23 and determines the number of times of photographing N for the same divided regions (the regions to be photographed) 500 in the biological tissue specimen and a limited range (Min and Max) of the RGB values based on the type of the staining reagent for the biological tissue specimen and the minimum values and the maximum values of the RGB values (step S505) .

Since step S506 and step S507 are the same as step S106 and step S107 in the first embodiment illustrated in FIG. 6, explanation of the steps is omitted here.

Since step S508 is the same as step S409 in the fourth embodiment illustrated in FIG. 18, explanation of the step is omitted here.

Then, the image processing system 10 can execute correction by limiting the range of the RGB values of the composite image 404 for each of the colors according to Formula (8) (step 509). In Formula (8), OutImageR (x, y), OutImageG (x, y), and OutImageB (x, y) are added-up values of the RGB values for each of the same pixels of the N secondary images 402, that is, the RGB values for each of the pixels of the composite image 404. Max R, Max G, Max B, Min R, Min G, and Min B indicate limited ranges of the RGB values. Further, CorrOutImageR (x, y), CorrOutImageG (x, y), and CorrOutImageB (x, y) are RGB values for each of pixels of an image after the correction (not illustrated). In the present embodiment, it is possible to display a tissue of attention in a high-contrast state by performing the correction explained above based on the limited range (Min and Max) of the RGB values.

$$\begin{matrix}C o r r O u t I m a g e R \left(x , y\right) = M i n \left(O u t I m a g e R \left(x , y\right) , M a x R\right) - M i n R \\ C o r r O u t I m a g e G \left(x , y\right) = M i n \left(O u t I m a g e G \left(x , y\right) , M a x G\right) - M i n G \\ C o r r O u t I m a g e B \left(x , y\right) = M i n \left(O u t I m a g e B \left(x , y\right) , M a x B\right) - M i n B\end{matrix}$$

Since step S510 is the same as step S109 in the first embodiment illustrated in FIG. 6, explanation of the step is omitted here.

As explained above, in the present embodiment, the RGB values in a suitable range are cut out based on the limited range (Min and Max) of the RGB values, that is, the range of the RGB values of the composite image 404 for each of colors is limited, whereby the tissue of attention can be displayed in a high-contrast state.

### 6.3 Modification

In the embodiment explained above, the type of the staining reagent is determined based on the shapes of the histograms of the RGB values. However, in the present embodiment, the type of the staining reagent is not limited to this. For example, the type of the staining reagent may be manually input by the user or the type of the staining reagent may be acquired from incidental information described on a label (not illustrated) stuck to the slide 300 using the thumbnail camera 110 explained above.

### 7. Summary

As explained above, in the embodiments of the present disclosure, by adding up a plurality of secondary images, it is possible to expand a dynamic range while reducing noise and eliminating missing of information due to insufficient gradation (discontinuity). Therefore, according to the embodiments of the present disclosure, a clear digital image of a biological tissue specimen can be acquired. Further, according to the embodiments of the present disclosure, since it is not requested to provide a multi-spectral sensor, an increase in manufacturing cost and an increase in the size of the image processing system 10 can be avoided. That is, according to the embodiments of the present disclosure, it is possible to acquire a clear digital image of a biological tissue specimen while avoiding an increase in manufacturing cost and an increase in size.

Further, in the embodiments of the present disclosure, the histograms of the pixel values are created in order to obtain the number of times of photographing N and the correction value. However, the histograms may be omitted and the minimum values and the maximum values of the pixel values may be directly obtained. In this way, an image processing time can be reduced. In the embodiments of the present disclosure, the primary image 400 and the secondary image 402 are stored in the storage unit 250 such as an HDD. However, the present disclosure is not limited to this. By using a memory incorporated in a GPU (Graphics Processing Unit) or a DSP (Digital Signal Processor), storage and addition may be simultaneously performed to reduce the image processing time.

Note that, in the embodiments of the present disclosure explained above, the photographing target is not limited to the biological tissue specimen and may be a fine mechanical structure or the like and is not particularly limited. The embodiments of the present disclosure explained above are not limited to be applied to a use such as medical or research use and is not particularly limited if the embodiments are applied to a use in which it is requested to perform highly accurate analysis and extraction using a high-contrast image.

### 8. Application Example

The technique according to the present disclosure can be applied to various products. For example, the technique according to the present disclosure may be applied to a pathological diagnosis system, a support system for the pathological diagnosis system, or the like (hereinafter referred to as a diagnosis support system) with which a doctor or the like diagnoses a lesion by observing cells or tissues collected from a patient. The diagnosis support system may be a WSI (Whole Slide Imaging) system that diagnoses a lesion or supports the diagnosis based on an image acquired using a digital pathology technique.

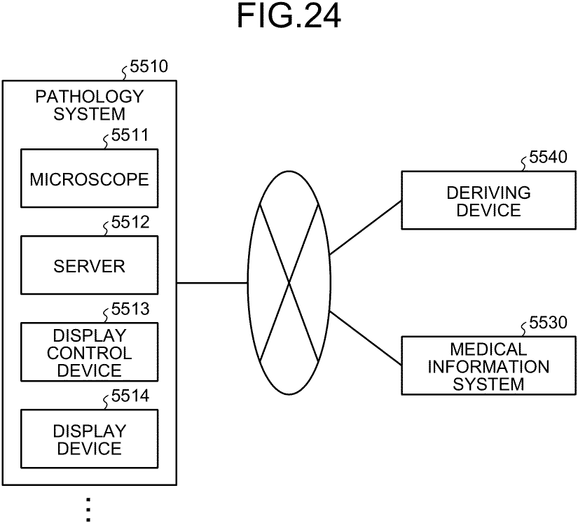

FIG. 24 is a diagram illustrating an example of a schematic configuration of a diagnosis support system 5500 to which the technique according to the present disclosure is applied. As illustrated in FIG. 24, the diagnosis support system 5500 includes one or more pathology systems 5510. Further, the diagnosis support system 5500 may include a medical information system 5530 and a deriving device 5540.

Each of the one or more pathology systems 5510 is a system mainly used by a pathologist and is introduced into, for example, a laboratory or a hospital. The pathology systems 5510 may be introduced into hospitals different from one another and are respectively connected to the medical information system 5530 and the deriving device 5540 via various networks such as a WAN (Wide Area Network) (including the Internet), a LAN (Local Area Network), a public line network, and a mobile communication network.

Each of the pathology systems 5510 includes a microscope (specifically, a microscope used in combination with a digital imaging technique) 5511, a server 5512, a display control device 5513, and a display device 5514.

The microscope 5511 has a function of an optical microscope and photographs an observation target stored in a glass slide to acquire a pathological image, which is a digital image. The observation target is, for example, a tissue or a cell collected from a patient and may be a piece of meat of an organ, saliva, blood, or the like. For example, the microscope 5511 functions as the DPI scanner 100 according to the first embodiment of the present disclosure.

The server 5512 stores and saves the pathological image acquired by the microscope 5511 in a not-illustrated storage unit. When accepting a browsing request from the display control device 5513, the server 5512 retrieves a pathological image from the not-illustrated storage unit and sends the retrieved pathological image to the display control device 5513. For example, the server 5512 functions as the image processing apparatus 200 according to the first embodiment of the present disclosure.

The display control device 5513 transmits a request for viewing the pathological image received from the user to the server 5512. Then, the display control device 5513 displays the pathological image received from the server 5512 on the display device 5514 in which liquid crystal, electro-luminescence (EL), a cathode ray tube (CRT), or the like is used. Note that the display device 5514 may be adapted to 4K or 8K, is not limited to one device, and may be a plurality of devices.

Here, when the observation target is a solid material such as a piece of meat of an organ, the observation target may be, for example, a stained thin slice. The thin slice may be produced, for example, by slicing a block piece cut out from a specimen such as an organ. In the slicing, the block piece may be fixed by paraffin or the like.

For staining of a thin slice, various types of staining may be applied, such as general staining showing the morphology of a tissue such as HE (Hematoxylin-Eosin) staining or immunostaining showing an immune state of a tissue such as IHC (Immunohistochemistry) staining. At that time, one thin slice may be stained using a plurality of different reagents or two or more thin slices (also referred to as adjacent thin slices) continuously cut out from the same block piece may be stained using different reagents.

The microscope 5511 can include a low-resolution photographing unit for photographing at low resolution and a high-resolution photographing unit for photographing at high resolution. The low-resolution photographing unit and the high-resolution photographing unit may be different optical systems or may be the same optical system. In the case of the same optical system, the resolution of the microscope 5511 may be changed according to a photographing target.

The glass slide storing the observation target is placed on a stage located within an angle of view of the microscope 5511. First, the microscope 5511 acquires an entire image within the angle of view using the low-resolution photographing unit and specifies a region of the observation target from the acquired entire image. Subsequently, the microscope 5511 divides a region where the observation target is present into a plurality of divided regions having a predetermined size and sequentially photographs images of the divided regions with the high-resolution photographing unit to acquire high-resolution images of the divided regions. In switching the target divided region, the stage may be moved, the photographing optical system may be moved, or both of the stage and the photographing optical system may be moved. The divided regions may overlap the divided regions adjacent thereto in order to prevent, for example, occurrence of a photographing omission region due to unintended slip of the glass slide. Further, the entire image may include identification information for associating the entire image and the patient. The identification information may be, for example, a character string or a QR code (registered trademark).

The high-resolution image acquired by the microscope 5511 is input to the server 5512. The server 5512 divides the high-resolution image into partial images (hereinafter referred to as tile images) having a smaller size. For example, the server 5512 divides one high-resolution image into 10 × 10 one hundred tile images in total. At this time, if the adjacent divided regions overlap, the server 5512 may apply stitching processing to high-resolution images adjacent to one another using a technique such as template matching. In that case, the server 5512 may divide an entire high-resolution image stuck together by the stitching processing to generate tile images. However, the generation of the tile images from the high-resolution image may be performed before the stitching processing.

The server 5512 can generate tile images having a smaller size by further dividing the tile images. The generation of such tile images may be repeated until tile images having a size set as a minimum unit are generated.

When the tile images of the minimum unit are generated in this way, the server 5512 executes, on all the tile images, tile synthesis processing for combining a predetermined number of adjacent tile images to generate one tile image. This tile synthesis processing may be repeated until one tile image is finally generated. By such processing, a tile image group having a pyramid structure in which layers are configured by one or more tile images is generated. In this pyramid structure, the numbers of pixels a tile image in a certain layer and a tile image in a layer different from this layer are the same. However, resolutions the tile images are different. For example, when 2 × 2 four tile images in total are combined to generate one tile image in an upper layer, the resolution of the tile image in the upper layer is ½ times the resolution of a tile image in a lower layer used for the combination.

By constructing the tile image group having such a pyramid structure, it is possible to switch a detail degree of the observation target displayed on the display device depending on a layer to which a display target tile image belongs. For example, when a tile image in the bottom layer is used, a narrow region of the observation target can be displayed in detail and a wider region of the observation target can be displayed coarser as a tile image in an upper layer is used.

The generated tile image group having the pyramid structure is stored in a not-illustrated storage unit together with, for example, identification information (referred to as tile identification information) that can uniquely identify the tile images. When receiving an acquisition request for a tile image including tile identification information from another device (for example, the display control device 5513 or the deriving device 5540), the server 5512 transmits the tile image corresponding to the tile identification information to the other device.

Note that the tile image, which is the pathological image, may be generated for each of photographing conditions such as a focal length and a staining condition. When the tile image is generated for each of the photographing conditions, another pathological image that corresponds to a photographing condition different from the specific photographing condition and is in the same region as the specific pathological image may be displayed side by side with the specific pathological image. The specific photographing condition may be designated by a viewer. When a plurality of photographing conditions are designated by the viewer, pathological images in the same region corresponding to the photographing conditions may be displayed side by side.

The server 5512 may store the tile image group having the pyramid structure in another storage device other than the server 5512, for example, a Cloud server. Further, a part or all of the entire tile image generation processing explained above may be executed by the Cloud server or the like.

The display control device 5513 extracts a desired tile image from the tile image group of the pyramid structure according to the input operation from the user and outputs the desired tile image to the display device 5514. With such processing, the user can obtain a feeling as if the user is observing the observation target while changing observation magnification. That is, the display control device 5513 functions as a virtual microscope. Virtual observation magnification here is actually equivalent to resolution.

Note that any method may be used as a method of photographing a high-resolution image. The divided regions may be photographed while repeatedly stopping and moving the stage to acquire a high-resolution image or the divided regions may be photographed while moving the stage at predetermined speed to acquire a high-resolution image on a strip. Furthermore, the processing for generating tile images from a high-resolution image is not an essential configuration. An image in which resolution changes stepwise may be generated by changing, stepwise, the resolution of the entire high-resolution image stuck together by the stitching processing. Even in this case, it is possible to present a low-resolution image in a wide area to a high-resolution image in a narrow area to the user stepwise.

The medical information system 5530 is a so-called electronic medical record system and stores information related to diagnosis such as information for identifying a patient, patient disease information, examination information and image information used for the diagnosis, a diagnosis result, and prescription medicine. For example, a pathological image obtained by photographing an observation target of a certain patient can be displayed on the display device 5514 by the display control device 5513 after being once stored via the server 5512. The pathologist using the pathology system 5510 performs pathology diagnosis based on a pathology image displayed on the display device 5514. A result of the pathological diagnosis performed by the pathologist is stored in the medical information system 5530.

The deriving device 5540 can perform analysis on the pathological image. For this analysis, a learning model created by machine learning can be used. The deriving device 5540 may derive a classification result of a specific region, an identification result of a tissue, and the like as an analysis result. Further, the deriving device 5540 may derive identification results such as cell information, a number, a position, and luminance information, scoring information for the identification results, and the like. These pieces of information derived by the deriving device 5540 may be displayed on the display device 5514 of the pathology system 5510 as diagnosis support information.

Note that the deriving device 5540 may be a server system configured by one or more servers (including a Cloud server) or the like. The deriving device 5540 may be configured to be built in, for example, the display control device 5513 or the server 5512 in the pathology system 5510. That is, various analyses on the pathological image may be executed in the pathology system 5510.

The technique according to the present disclosure can be suitably applied to the server 5512 as explained above among the components explained above. Specifically, the technique according to the present disclosure can be publicly applied to image processing in the server 5512. By applying the technique according to the present disclosure to the server 5512, a clearer pathological image can be obtained. Therefore, diagnosis of a lesion can be more accurately performed.

Note that the configuration explained above can be applied not only to the diagnosis support system but also to all biological microscopes such as a confocal microscope, a fluorescence microscope, and a video microscope in which a digital imaging technology is used. Here, the observation target may be a biological sample such as a cultured cell, a fertilized egg, or a sperm, a biological material such as a cell sheet or a three-dimensional cell tissue, or an organism such as a zebrafish or a mouse. The observation target can be observed in a state of being stored not only in a glass slide and but also in a well plate, a petri dish, or the like.

Further, a moving image may be generated from a still image of the observation target acquired using a microscope in which the digital imaging technology is used. For example, a moving image may be generated from still images continuously photographed for a predetermined period or an image sequence may be generated from still images photographed at predetermined intervals. By generating the moving image from the still images in this way, it is possible to analyze dynamic characteristics of the observation target such as movement such as pulsation, elongation, and migration of cancer cells, nerve cells, myocardial tissue, sperm, and the like and a division process of cultured cells and fertilized eggs using machine learning.

### 9. Hardware Configuration

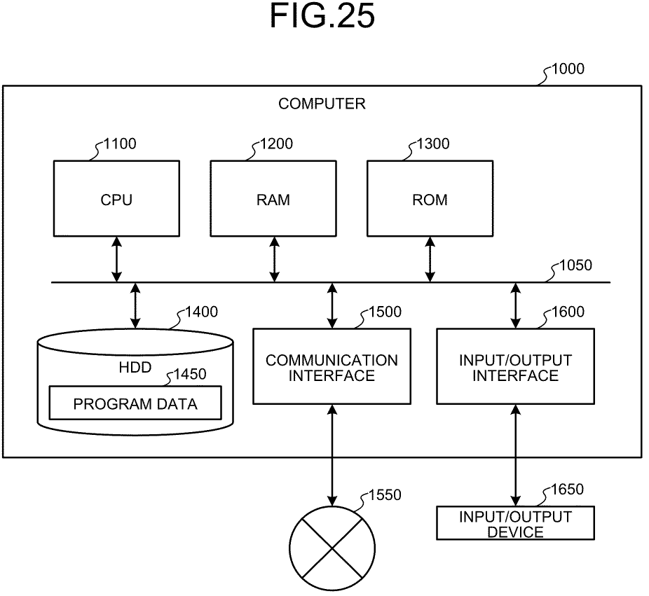

Information equipment such as the image processing apparatus 200 according to the embodiments explained above is realized by, for example, the computer 1000 having a configuration illustrated in FIG. 25. The image processing apparatus 200 according to the first embodiment is explained as an example. FIG. 25 is a hardware configuration diagram illustrating an example of the computer 1000 that realizes the functions of the image processing apparatus 200. The computer 1000 includes a CPU 1100, a RAM 1200, a ROM (Read Only Memory) 1300, a HDD (Hard Disk Drive) 1400, a communication interface 1500, and an input/output interface 1600. The units of the computer 1000 are connected by a bus 1050.

The CPU 1100 operates based on programs stored in the ROM 1300 or the HDD 1400 and controls the units. For example, the CPU 1100 develops, in the RAM 1200, the programs stored in the ROM 1300 or the HDD 1400 and executes processing corresponding to various programs.

The ROM 1300 stores a boot program such as a BIOS (Basic Input Output System) to be executed by the CPU 1100 at a start time of the computer 1000, a program depending on hardware of the computer 1000, and the like.

The HDD 1400 is a computer-readable recording medium that non-transiently records a program to be executed by the CPU 1100, data used by such a program, and the like. Specifically, the HDD 1400 is a recording medium that records an information processing program according to the present disclosure, which is an example of program data 1450.

The communication interface 1500 is an interface for the computer 1000 to be connected to an external network 1550 (for example, the Internet). For example, the CPU 1100 receives data from other equipment and transmits data generated by the CPU 1100 to the other equipment via the communication interface 1500.

The input/output interface 1600 is an interface for connecting an input/output device 1650 and the computer 1000. For example, the CPU 1100 receives data from an input device such as a keyboard or a mouse via the input/output interface 1600. The CPU 1100 transmits data to an output device such as a display, a speaker, or a printer via the input/output interface 1600. The input/output interface 1600 may function as a media interface that reads a program or the like recorded in a predetermined computer-readable recording medium (a medium). The medium is, for example, an optical recording medium such as a DVD (Digital Versatile Disc) or a PD(Phase change rewritable Disk), a magneto-optical recording medium such as an MO (Magneto-Optical Disk), a tape medium, a magnetic recording medium, or a semiconductor memory.

For example, when the computer 1000 functions as the image processing apparatus 200 according to the first embodiment, the CPU 1100 of the computer 1000 executes an image processing program loaded on the RAM 1200 to thereby realize the functions of the first acquisition unit 212, the image range acquisition unit 214, the number-of-times determination unit 216, the scanner control unit 218, the correction value determination unit 220, the second acquisition unit 222, the combining unit 224, and the like. The HDD 1400 may store the image processing program according to the present disclosure and data in the storage unit 250. Note that the CPU 1100 reads the program data 1450 from the HDD 1400 and executes the program data 1450. However, as another example, the CPU 1100 may acquire the image processing program from another device via the external network 1550.

### 10. Supplement

Note that the embodiments of the present disclosure explained above can include, for example, an image processing method executed in the image processing apparatus or the image processing system explained above, a program for causing the image processing apparatus to function, and a non-transitory tangible medium in which the program is recorded. The program may be distributed via a communication line (including wireless communication) such as the Internet.

The steps in the processing method in the embodiments of the present disclosure explained above may not always be processed according to the described order. For example, the steps may be processed with the order changed as appropriate. The steps may be partially processed in parallel or individually instead of being processed in time series. Further, the processing of the steps may not always be processed according to the described method and may be processed by, for example, another functional unit according to another method.

Among the kinds of processing explained in the above embodiments, all or a part of the processing explained as being automatically performed can be manually performed or all or a part of the processing explained as being manually performed can be automatically performed by a publicly-known method. Besides, the processing procedure, the specific names, and the information including the various data and parameters explained in the document and illustrated in the drawings can be optionally changed except when specifically noted otherwise. For example, the various kinds of information illustrated in the figures are not limited to the illustrated information.

The illustrated components of the devices are functionally conceptual and are not always required to be physically configured as illustrated in the figures. That is, specific forms of distribution and integration of the devices are not limited to the illustrated forms and all or a part thereof can be configured by being functionally or physically distributed and integrated in any unit according to various loads, usage situations, and the like.

The preferred embodiments of the present disclosure are explained in detail above with reference to the accompanying drawings. However, the technical scope of the present disclosure is not limited to such examples. It is evident that those having the ordinary knowledge in the technical field of the present disclosure can arrive at various alterations or corrections within the category of the technical idea explained in claims. It is understood that these alterations and corrections naturally belong to the technical scope of the present disclosure.

The effects described in this specification are only explanatory or illustrative and are not limiting. That is, the technique according to the present disclosure can achieve other effects obvious for those skilled in the art from the description of this specification together with the effects described above or instead of the effects.

Note that the present technique can also take the following configurations.

An image processing apparatus comprising:
a first pixel signal acquisition unit that acquires a first pixel signal by photographing of a region to be photographed of a biological tissue specimen;
a range acquisition unit that acquires a pixel value range in the first pixel signal;
a number-of-times determination unit that determines a number of times of photographing for at least a part of the region to be photographed of the biological tissue specimen based on the pixel value range;
a second pixel signal acquisition unit that acquires a second pixel signal, which is a pixel signal of at least a part of the region to be photographed of the biological tissue specimen, the second pixel signal being obtained by being photographed according to the number of times of photographing; and
an image generation unit that generates an output image based on at least a part of a plurality of the second pixel signals.

The image processing apparatus according to (1), further comprising a correction value determination unit that determines a correction value based on the pixel value range, wherein

the image generation unit executes correction on the second pixel signal or the output image using the correction value.

The image processing apparatus according to (2), further comprising a region determination unit that determines one or a plurality of the regions to be photographed in the biological tissue specimen based on a photographed image related to the first pixel signal, wherein

the range acquisition unit acquires the pixel value range in a pixel signal of the region to be photographed included in the first pixel signal.

The image processing apparatus according to (3), wherein the region determination unit determines the region to be photographed using an image recognition model obtained by machine learning.

The image processing apparatus according to (2), wherein the range acquisition unit acquires a maximum value and a minimum value of the first pixel signal.

The image processing apparatus according to (5), wherein the range acquisition unit generates a histogram of the first pixel signal.

The image processing apparatus according to (5) or (6), wherein
the number-of-times determination unit refers to a predetermined table stored in advance and determines the number of times of photographing based on the minimum value and the maximum value, and
the correction value determination unit refers to the predetermined table and determines the correction value based on the minimum value.

The image processing apparatus according to (7), wherein the predetermined table is generated in advance based on a photographed image quality model obtained by machine-learning a photographing history in past.

The image processing apparatus according to (7) or (8), wherein the number-of-times determination unit selects the predetermined table in use based on information given to the biological tissue specimen.

The image processing apparatus according to any one of (5) to (9), wherein the first pixel signal is luminance and chroma of pixels converted into an HLS signal or a YCC signal.

The image processing apparatus according to (5) or (6), wherein
the number-of-times determination unit calculates the number of times of photographing based on the minimum value and the maximum value,
the correction value determination unit calculates the correction value based on the minimum value, and
the image generation unit executes the correction by subtracting the correction value from the second pixel signal.

The image processing apparatus according to (11), wherein the first pixel signal is a level value for each of colors of pixels.

The image processing apparatus according to any one of (1) to (12), wherein the image generation unit adds up level values for each of colors of pixels of a plurality of the second pixel signals.

The image processing apparatus according to (1), further comprising a condition determination unit that determines a photographing condition for at least a part of the region to be photographed of the biological tissue specimen based on the pixel value range, wherein

the second pixel signal acquisition unit acquires the second pixel signal, which is a pixel signal of at least a part of the region to be photographed of the biological tissue specimen, by photographing according to the imaging condition.

The image processing apparatus according to (14), wherein the photographing condition includes a condition for at least one of irradiation intensity, a wavelength of irradiation light, and an exposure time.

The image processing apparatus according to any one of (1) to (15), wherein the biological tissue specimen is a biological tissue specimen stained by one or more staining reagents.

The image processing apparatus according to (1), wherein
the biological tissue specimen is a biological tissue specimen stained with one or a plurality of staining reagents,
the image processing apparatus further comprises a specifying unit that specifies a type of the staining reagent, and
the number-of-times determination unit determines the number of times of photographing based on the type of the staining reagent.

The image processing apparatus according to (17), wherein the specifying unit specifies the type of the staining reagent based on the pixel value range.

The image processing apparatus according to (17) or (18), further comprising a correction value determination unit that determines a correction value for correction for an output image based on the type of the staining reagent.

The image processing apparatus according to (19), wherein the image generation unit executes the correction by limiting a range of a pixel signal of the output image based on the correction value.

The image processing apparatus according to any one of (1) to (20), wherein a photographed image related to the first pixel signal has a wider angle of view or a same angle of view compared with a photographed image related to the second pixel signal.

The image processing apparatus according to any one of (1) to (20), wherein a photographed image related to the first pixel signal has lower resolution or same resolution compared with a photographed image related to the second pixel signal.

An image processing method comprising an image processing apparatus:
acquiring a first pixel signal by photographing of a region to be photographed of a biological tissue specimen;
acquiring a pixel value range in the first pixel signal;
determining a number of times of photographing for at least a part of the region to be photographed of the biological tissue specimen based on the pixel value range;
acquiring a second pixel signal, which is a pixel signal of at least a part of the region to be photographed of the biological tissue specimen, the second pixel signal being obtained by performing photographing according to the number of times of imaging; and
generating an output image based on at least part of a plurality of the second pixel signals.

An image processing system comprising:
an image processing apparatus that executes image processing; and
a program for causing the image processing apparatus to execute the image processing, wherein
the image processing apparatus includes:
a first pixel signal acquisition unit that acquires a first pixel signal by photographing of a region to be photographed of a biological tissue specimen;
a range acquisition unit that acquires a pixel value range in the first pixel signal;
a number-of-times determination unit that determines a number of times of photographing for at least a part of the region to be photographed of the biological tissue specimen based on the pixel value range;
a second pixel signal acquisition unit that acquires a second pixel signal, which is a pixel signal of at least a part of the region to be photographed of the biological tissue specimen, the second pixel signal being obtained by being photographed according to the number of times of photographing; and
an image generation unit that generates an output image based on at least a part of a plurality of the second pixel signals.

REFERENCE SIGNS LIST

| 10, 10a, 10b | INFORMATION PROCESSING SYSTEM |
|---|---|
| 100 | DPI SCANNER |
| 102, 112, 122 | LIGHT SOURCE UNIT |
| 104, 114, 124 | SENSOR UNIT |
| 106, 116, 126 | CONTROL UNIT |
| 108 | STAGE |
| 110 | THUMBNAIL CAMERA |
| 120 | MAIN CAMERA |
| 130 | SLIDE LOADER |
| 200, 200a, 200b, 200c, 200d | IMAGE PROCESSING APPARATUS |
| 210, 210a, 210b, 210c, 210d | PROCESSING UNIT |
| 212 | FIRST ACQUISITION UNIT |
| 214, 214a, 214b | IMAGE RANGE ACQUISITION UNIT |
| 216, 216a, 216b, 216d | NUMBER-OF-TIMES DETERMINATION UNIT |
| 218, 218c | SCANNER CONTROL UNIT |
| 220, 220a, 220b, 220d | CORRECTION VALUE DETERMINATION UNIT |
| 222 | SECOND ACQUISITION UNIT |
| 224, 224a, 224c | COMBINING UNIT |
| 226, 228 | HLS SIGNAL GENERATION UNIT |
| 230 | RGB SIGNAL GENERATION UNIT |
| 232 | STITCHING UNIT |
| 234 | CONDITION DETERMINATION UNIT |
| 236 | DETERMINATION UNIT |
| 238 | CORRECTION UNIT |
| 240 | COMMUNICATION UNIT |
| 250 | STORAGE UNIT |
| 252, 254, 256 | TABLE |
| 260 | DISPLAY UNIT |
| 300 | SLIDE |
| 400, 410 | PRIMARY IMAGE |
| 402, 412 | SECONDARY IMAGE |
| 404 | COMPOSITE IMAGE |
| 500 | DIVIDED REGION |

1. An image processing apparatus comprising:
a first pixel signal acquisition unit that acquires a first pixel signal by photographing of a region to be photographed of a biological tissue specimen;
a range acquisition unit that acquires a pixel value range in the first pixel signal;
a number-of-times determination unit that determines a number of times of photographing for at least a part of the region to be photographed of the biological tissue specimen based on the pixel value range;
a second pixel signal acquisition unit that acquires a second pixel signal, which is a pixel signal of at least a part of the region to be photographed of the biological tissue specimen, the second pixel signal being obtained by being photographed according to the number of times of photographing; and
an image generation unit that generates an output image based on at least a part of a plurality of the second pixel signals.

2. The image processing apparatus according to claim 1, further comprising a correction value determination unit that determines a correction value based on the pixel value range, wherein
the image generation unit executes correction on the second pixel signal or the output image using the correction value.

3. The image processing apparatus according to claim 2, further comprising a region determination unit that determines one or a plurality of the regions to be photographed in the biological tissue specimen based on a photographed image related to the first pixel signal, wherein
the range acquisition unit acquires the pixel value range in a pixel signal of the region to be photographed included in the first pixel signal.

4. The image processing apparatus according to claim 3, wherein the region determination unit determines the region to be photographed using an image recognition model obtained by machine learning.

5. The image processing apparatus according to claim 2, wherein the range acquisition unit acquires a maximum value and a minimum value of the first pixel signal.

6. The image processing apparatus according to claim 5, wherein the range acquisition unit generates a histogram of the first pixel signal.

7. The image processing apparatus according to claim 5, wherein
the number-of-times determination unit refers to a predetermined table stored in advance and determines the number of times of photographing based on the minimum value and the maximum value, and
the correction value determination unit refers to the predetermined table and determines the correction value based on the minimum value.

8. The image processing apparatus according to claim 7, wherein the predetermined table is generated in advance based on a photographed image quality model obtained by machine-learning a photographing history in past.

9. The image processing apparatus according to claim 7, wherein the number-of-times determination unit selects the predetermined table in use based on information given to the biological tissue specimen.

10. The image processing apparatus according to claim 5, wherein the first pixel signal is luminance and chroma of pixels converted into an HLS signal or a YCC signal.

11. The image processing apparatus according to claim 5, wherein
the number-of-times determination unit calculates the number of times of photographing based on the minimum value and the maximum value,
the correction value determination unit calculates the correction value based on the minimum value, and
the image generation unit executes the correction by subtracting the correction value from the second pixel signal.

12. The image processing apparatus according to claim 11, wherein the first pixel signal is a level value for each of colors of pixels.

13. The image processing apparatus according to claim 1, wherein the image generation unit adds up level values for each of colors of pixels of a plurality of the second pixel signals.

14. The image processing apparatus according to claim 1, further comprising a condition determination unit that determines a photographing condition for at least a part of the region to be photographed of the biological tissue specimen based on the pixel value range, wherein
the second pixel signal acquisition unit acquires the second pixel signal, which is a pixel signal of at least a part of the region to be photographed of the biological tissue specimen, by photographing according to the imaging condition.

15. The image processing apparatus according to claim 14, wherein the photographing condition includes a condition for at least one of irradiation intensity, a wavelength of irradiation light, and an exposure time.

16. The image processing apparatus according to claim 1, wherein the biological tissue specimen is a biological tissue specimen stained by one or more staining reagents.

17. The image processing apparatus according to claim 1, wherein
the biological tissue specimen is a biological tissue specimen stained with one or a plurality of staining reagents,
the image processing apparatus further comprises a specifying unit that specifies a type of the staining reagent, and
the number-of-times determination unit determines the number of times of photographing based on the type of the staining reagent.

18. The image processing apparatus according to claim 17, wherein the specifying unit specifies the type of the staining reagent based on the pixel value range.

19. The image processing apparatus according to claim 17, further comprising a correction value determination unit that determines a correction value for correction for an output image based on the type of the staining reagent.

20. The image processing apparatus according to claim 19, wherein the image generation unit executes the correction by limiting a range of a pixel signal of the output image based on the correction value.

21. The image processing apparatus according to claim 1, wherein a photographed image related to the first pixel signal has a wider angle of view or a same angle of view compared with a photographed image related to the second pixel signal.

22. The image processing apparatus according to claim 1, wherein a photographed image related to the first pixel signal has lower resolution or same resolution compared with a photographed image related to the second pixel signal.

23. An image processing method comprising an image processing apparatus:
acquiring a first pixel signal by photographing of a region to be photographed of a biological tissue specimen;
acquiring a pixel value range in the first pixel signal;
determining a number of times of photographing for at least a part of the region to be photographed of the biological tissue specimen based on the pixel value range;
acquiring a second pixel signal, which is a pixel signal of at least a part of the region to be photographed of the biological tissue specimen, the second pixel signal being obtained by performing photographing according to the number of times of imaging; and
generating an output image based on at least part of a plurality of the second pixel signals.

24. An image processing system comprising:
an image processing apparatus that executes image processing; and
a program for causing the image processing apparatus to execute the image processing, wherein
the image processing apparatus includes:
a first pixel signal acquisition unit that acquires a first pixel signal by photographing of a region to be photographed of a biological tissue specimen;
a range acquisition unit that acquires a pixel value range in the first pixel signal;
a number-of-times determination unit that determines a number of times of photographing for at least a part of the region to be photographed of the biological tissue specimen based on the pixel value range;
a second pixel signal acquisition unit that acquires a second pixel signal, which is a pixel signal of at least a part of the region to be photographed of the biological tissue specimen, the second pixel signal being obtained by being photographed according to the number of times of photographing; and
an image generation unit that generates an output image based on at least a part of a plurality of the second pixel signals.
